(19) United States
(12) Patent Application Publication
(10) Pub. No.: US 2025/0213342 A1
(43) Pub. Date: Jul. 3, 2025

(54) IMAGING A JOINT TO CALCULATE RANGE OF MOTION AND POSITIONING CHARACTERISTICS

(71) Applicant: Spinal Guides Labs, LLC, Carolina, PR (US)

(72) Inventors: Albert Y. Davydov, Carolina, PR (US); Gaurav Saxena, Indore (IN)

(21) Appl. No.: 19/002,554

(22) Filed: Dec. 26, 2024

Related U.S. Application Data
(60) Provisional application No. 63/615,828, filed on Dec. 29, 2023.

(51) Int. Cl. A61C 19/045; A61B 5/00; A61B 6/00; A61B 6/51; G16H 20/40; G16H 30/40; G16H 50/20

(52) U.S. Cl. CPC A61C 19/045; A61B 5/0077; A61B 6/51; A61B 6/5247; G16H 20/40; G16H 30/40; G16H 50/20

(57) ABSTRACT

Systems and methods are provided for calculating Range of Motion (ROM), movement, and positioning characteristics of a human joint using image processing. A method, according to one implementation, includes the step of arranging a clamp in a fixed position with respect to a first bone of a patient's joint, whereby the clamp has a reference object attached thereto. The method further includes the step of capturing images of the patient's joint and the reference object. Also, the method includes the step of receiving the captured images and monitoring characteristics of movements and positioning of the first bone of the patient's joint with respect to a second bone of the patient's joint. A system is provided to perform the above-described method.

### PRIORITY

This application claims priority to U.S. Provisional Application Ser. No. 63/615,828, filed Dec. 29, 2023, the contents of which are hereby incorporated by reference.

### TECHNICAL FIELD

The present disclosure relates generally to monitoring joints of the human body, and more particularly relates to systems and methods for monitoring the range of motion and positioning characteristics of, for example, the human jaw using imaging methodologies.

### BACKGROUND

The Temporomandibular Joints (TMJs) are the joints that connect the mandible (i.e., lower jaw) to the temporal bone at the skull. There are, of course, two TMJs—one TMJ on each side of the lower jaw. The TMJs allow the mandible to rotate, slide side-to-side, and slide forward and backward with respect to the maxilla. The positioning between the mandible and maxilla is referred to as the “maxillomandibular relationship,” “mandibular maxillary relationship,” or “jaw relation” and refers to the position of the mandible with respect to the maxilla. In dentistry, oral surgery, facial surgery, etc., the maxillomandibular relationship can be used for reconstructive surgery, dental implants, creation of dentures, etc.

The TMJ is the most complex joint in the body consisting of three bones on each side rigidly connected with the Mandible and, if evaluated paired, consisting of 5 bones: L Temporal Bone, L Temporomandibular Disk, R Temporal Bone, R Temporomandibular Disk, and Mandible consisting of left and right sides fused in the symphysis in the chin area which provides Left and Right Condylar processes for respective TMJs. Maxilla is a paired bone fused to Skull opposing the Mandible on both, left and right sides. Mandibular and Maxillary teeth oppose each other providing occlusal table and their relationship affects dramatically the lower third of facial height, as well the facial esthetics.

Different types of TMJ apparatuses may be used to study positioning and kinematics of the TMJ and may include different types of devices and methods for fixing instruments to the head, face, teeth, or jaw. These fixation devices may serve the purpose of providing the secure placement of sensors for measuring aspects of the teeth, jaw, face, etc. The fixation devices may include headbands, headgear, straps, etc., some of which can be circular in shape, may tighten on top of or behind the head, and may employ supportive elements for TMJ/maxillofacial study apparatuses.

For example, there are systems that can be positioned in the front of the head or face and may resemble glasses, like 3D video viewing apparatuses. Also, some systems may use dental facebows that provide a way to mount sensors in front of the face and are configured to transfer mechanical data to dental articulators, which are physical devices used to simulate the human jaw.

However, existing headbands, headgear, and fixation systems possess an inherent lack of precision and reliability because they are essentially positioned arbitrarily. For instance, at the time of locking the instruments in place, the soft tissue of the human face may naturally contract during tightening or closure of the gear. Also, when an apparatus is locked in place, human skin may tend to experience turgor changes, and therefore, the position of the instruments may change each time they are fixed to the patient or when more pressure is applied to the skin.

Some equipment for monitoring the maxillomandibular relationship may even include electronic instruments. When these systems are loaded with different electronics, they can become heavy and, therefore, may become unstable and fall off of the patient's head, especially if the person is required to move his or her head forward, backward, or to the side. Also, electronic positioning measuring equipment may provide inaccurate results in the presence of magnetic fields.

FIG. 1 is a front view of a conventional system 10 attached to the head of a patient 12 for monitoring the positioning or Range of Motion (ROM) of the mandible (or lower jaw) of the patient 12 relative to the maxilla (or upper jaw). The system 10 may be formed as a digital dental facebow, which is attached externally to the face and mouth of the patient 12 to measure functional and/or aesthetic characteristics of the mouth, teeth, jaw, etc. The system 10 includes a bow 14, which is positioned on the head, and an intraoral sensor plate 16, which includes an intraoral fork 18 (e.g., U-shaped fork) and a sensor 20. The sensor 20 may include electronic sensing elements configured to transfer data to a computer (not shown) or to a fully adjustable dental articulator (not shown). In additional to these electronic systems, some traditional mechanical facebows may be used for the same function as digital ones except they are not represented as high technology tools.

Hands 21 of the doctor (e.g., dentist, oral surgeon, etc.) are shown in FIG. 1. The doctor can place the various pieces of the system 10 on the head of the patient 12. Also, the doctor may use his hands 21 to steady the head of the patient 12 during testing and assist the patient 12 opening and closing the mouth for proper positioning, which can enable more accurate monitoring.

As attached to the head of the patient 12, the bow 14 can be passively engaged on the head while exerting a very minimal pressure or grip. The sensor 20 may be positioned on the intraoral fork 18. The system 10 (or facebow device) is used to measure the relationship between the upper jaw and the skull of the patient 12 with intentions to transfer records to a fully adjustable digital dental or mechanical articulator.

The intraoral fork 18 and the sensor 20 mounted thereon are both of a considerable size, and therefore have limitations and disadvantages for use in studying the physiological joint movements. For example, the intraoral fork 18 with the sensor 20 cannot be used to measure dynamic physiological movements or the speed of opening and closing of one's jaw, chewing movements, etc. As is known, the system 10 is primarily used to program a fully adjustable articulator for the purposes of designing, creating, and/or selecting prosthetic teeth.

In addition, the system 10 cannot be used for patients having edentulous mouths (i.e., patients having no teeth), as well in the mouths with deep bites (i.e., patients with misalignment of the upper teeth to the lower teeth), as the system 10 cannot be placed onto the teeth due to anatomical obstacles. That is, in edentulous jaws, there are no teeth on which to put the intraoral fork 18, and, in the case of a deep bite, there is no space in between the upper and lower teeth due to lack of space between the upper and lower jaws.

One of the goals of programming a dental articulator may be to remove interferences on the anatomical teeth for both “working” teeth (i.e., teeth of the side of the mouth where chewing normally occurs) and “non-working” teeth (i.e., teeth of the side of the mouth used for balancing chewing actions). These and other movement actions may be considered during a prosthetics fabrication process.

It is known that the patient 12 can function better with such a prosthesis. The patient's bone level will be more preserved from loss with such a prosthesis and the chair time to adjust the prosthesis in the mouth during clinical visits of delivery of the prosthesis can be reduced.

As above, dental facebows are used during partial, or full denture reconstruction to program fully adjustable articulators to select proper occlusal guidance, working and non-working side relationships, and proper selection of artificial teeth.

FIG. 2 shows a skull 22 of the patient 12 on the left side of the drawing. On the right side of the drawing, FIG. 2 shows the patient 12 with the system 10 or facebow attached. The hands 21 of the doctor in FIG. 2 may help to position the mandible of the patient 12 in an opened orientation. Also, the doctor may support the intraoral sensor plate 16 while the patient 12 is moving his jaw and the sensor 20 is configured to record tracings 24 of the mandible with respect to the maxilla. If the doctor does not guide the patient's jaw during natural movements, the intraoral sensor plate 16 would normally be dislocated, which would result in bad data.

There is an inherent movement of the head while opening and closing of the jaws during any type of monitoring. Since the conventional systems do not use reference points on the head that are fixed, these technologies do not provide means for compensation due to such head movements. Without head compensation, the results taken from the electronic sensors are usually inconsistent and erroneous. Therefore, there is a need in the field of maxillomandibular relationship detection to incorporate reference points in the detections and calculations of ROM and positioning of the jaws.

Other devices in the US market, like K7 from Myotronics, or Jaw Tracker from Bioresearch Associates utilize bulky and cumbersome equipment, all of which have headgear and sensors on the headgear with sensor magnets attached with a glue on the patients' lower front teeth. These systems have questionable precision and reliability because of placement, displacement, and replacement issues with these systems. In the conventional systems, sensors are typically not rigidly connected to headgears and minute movements of the head in the Cervicocranium are not considered, thereby making the positioning and repositioning arbitrary and inconsistent. Therefore, the reliability of the data obtained from these conventional systems is often questionable.

Also, the conventional methods do not work if the patient has no front teeth on the lower jaw to which the magnet can be attached or when there is simply no space for a magnet to be attached in between the upper and lower jaws (e.g., for deep bite conditions). The magnet sensor must not be autoclaved as heat can destroy the magnet. Therefore, it can only be sanitized, which can make the method unsafe or unsanitary with regard to infectious disease transmissions.

The conventional systems may use magnetic sensors intraorally via some kind of a glue attaching the magnet sensor to outside (facial, labial-side) of the lower teeth. The magnet sensor cannot be chemically sterilized as chemical sterilizers will normally destroy the sensor. The magnet sensor cannot be autoclaved since autoclaving will also destroy the sensor. In addition, conventional systems cannot provide reliable data in proximity of electromagnetic field since magnetic field will interfere with electromagnetic coils sensing magnetic sensors.

Also, Range of Motion (ROM) measurements do not normally allow for compensation due to head movements. The conventional systems also suffer from the arbitrary positioning of the headgear on the head. Also, the positioning of magnet sensors in the mouth and within an electromagnetic field is arbitrary.

Magnet sensors, over time, lose their strength making the data unreliable and there is no indicator available in the systems to identify the point at which the loss of magnet power is initiated anywhere on any of the conventional system.

Furthermore, the magnet sensors of conventional systems are usually exposed to biological fluid, which can be unsafe to use. This can lead to the spread of infections from one patient to another. Also, the use of magnet sensors provides a very imprecise record when used with lower full dentures, since stability of a full denture in the mouth upon full opening and closing is questionable.

Thus, because of the issues of conventional systems with respect to measuring or monitoring the positioning and ROM of a patient's jaw and cannot help in proper diagnosis of real-life scenarios, these systems often result in inaccurate or improper prosthetic fabrication, or at times, cannot be used at all. Therefore, a need exists for techniques for improved range of motion calculation and monitoring in positioning of mandible to skull in joint imaging for diagnosis and care, as described in the present disclosure with respect to the preferred embodiments discussed below.

### SUMMARY

Devices, systems, and methods for range of motion (ROM) calculation, study of temporomandibular kinematics and positioning in joint imaging are provided. According to one implementation, an apparatus includes a clamp arranged in a fixed position with respect to a first bone of a patient's joint, wherein the clamp has a reference object attached thereto. In this implementation, the apparatus further includes one or more cameras arranged to capture images of the patient's joint and the reference object. Also, the apparatus includes a processing device configured to receive the captured images from the one or more cameras and monitor characteristics of movements and positioning of the first bone of the patient's joint with respect to a second bone of the patient's joint.

According to some embodiments, the patient's joint may include one Temporomandibular Joint, which is rigidly connected to another Temporomandibular Joint on the other side via Mandibular Symphysis, where a first bone of the patient's joint is Mandible with its Condylar Process on each side, a second bone affecting the patient's joint as a barrier in closing, is a Maxilla, a third bone is a Temporal bone the lower portion of which articulates with the Disc and Mandibular Condyle forming the Joint in which one Mandibular Condyle joined on each side with a Temporomandibular Joint Disk, which is treated as a bone, the fourth bone, and where the characteristics of movements and positioning of the first bone with respect to the second bone is a maxillomandibular relationship.

The Temporomandibular Disc is situated between a Condylar Processus of Mandible and Fossa Articularis of Bone Temporale and is represented by a dense connective tissue supported by ligaments. It is treated as a bone. In front, it is important to note, that the disc has the Internal Pterygoid Muscle attachment, and behind the disc has retrodiscal tissues that are rich with pain receptors and blood vessels. During function of the TMJ, the disc provides a protective cushion for the tissues as forces of mastication may reach very high levels. In a normal, healthy joint, there are minimal vibrations, but depending on different medical conditions, or different injuries to the ligaments, or maxillofacial conditions the disc may lose its support from the ligaments, or its texture can change resulting an increase of movement of the disc inside the TMJ during function producing various vibrations. Depending on the locality of vibrations within functional cycles and their amplitudes and energy it is possible to diagnose various conditions affecting the disc and the joint. Once such a map is available to the dental professional, he/she is able to position the disc within the TMJ space using the system with precision.

In some embodiments, the processing device may be configured to create a baseline image including facial landmark points. The processing device may further use characteristics of the temporomandibulomaxillary relationship in the design of the most physiological dental prosthetics for the patient. Temporomandibulomaxillary relationship is a complex relationship between Mandible and Maxilla where Mandibular Condyles relate to Skull in the Temporal Fossa of Temporal Bone with certain position of the Temporomandibular Joint Disc in relation to the Fossa and the Condyle. This is the most desirable relationship to monitor to achieve the most physiologically acceptable dental prosthesis. Traditionally, in most of the dental schools, students were thought that the most desirable position of the mandible to the Skull is a maximum Mandibular retruded position because it is the one that is the most reproducible. This fundamentally incorrect approach is a source of multitude of iatrogenic problems in patients because of “one size that fits all” concept. This “most retruded” Mandibular position is used up until now because it is the “most reproducible”, and there is no device that would allow to precisely reproduce other “custom made” Mandibular—Maxillary-Skull, or temporomandibular-maxillary, relationships with precision and repeatability. Only with the use of the system and methods described here, these records are possible to obtain, they are practical and useful, as they are reliable and reproducible, infection disease transmission safe and interrater reliable.

The apparatus may further be defined whereby the reference object is configured as a color-coded spherical object. The clamp, for example, may include an upper prong, a shaft, and an adjustable lower prong, whereby the clamp may be configured to compress soft tissue surrounding the first bone to hold the clamp in a fixed relationship with the first bone. The apparatus may further comprise a display device connected to the processing device, wherein the display device may be configured to present a User Interface (UI) that shows images captured by the one or more cameras. The UI, for example, may further be configured to provide one or more selectable buttons allowing a user to select options for operating the apparatus.

Furthermore, the processing device may be configured to utilize Artificial Intelligence (AI) to monitor the characteristics of the movements and positioning of the first bone with respect to the second bone and the first bone and the Disk on each side. The apparatus may further include one or more of a vibration sensor and an audible capture device for detecting one or more of vibration, sound, and voice at the patient's joint related to movement of the first bone with respect to the second bone, the first bone and the disk on each side and the third bone, the Temporal Fossa, to further characterize the patient's joint. In addition, the processing device may be further configured to create a homography matrix based on a current image plane with respect to a reference image plane. Also, in some embodiments, the processing device may be configured to utilize the characteristics of the movements and positioning of the first bone with respect to the second bone, the first bone and the disk on each side and the third bone, the Temporal Fossa to create one or more graphs and contours.

According to an aspect of the present disclosure, an apparatus is provided including a clamp arranged in a fixed position with respect to a first bone of a patient's joint, the clamp having a reference object attached thereto; one or more cameras arranged to capture images of facial landmark points and the reference object; and a processing device configured to receive the captured images from the one or more cameras and monitor characteristics of movements and positioning of the first bone of the patient's joint with respect to a second bone of the patient's joint.

In one aspect, Temporomandibular Complex of the patient includes one or more Temporomandibular Joints (TMJs), the first bone of the patient's joint is a mandible, the second bone of the patient's joint is a maxilla, the third bone is Temporal Bone, the fourth bone is a Temporomandibular Disc and the characteristics of movements and positioning of the first bone with respect to the second bone and the Discs is temporomandibulomaxillary relationship.

In another aspect, the processing device is configured to create a baseline image including the facial landmark points.

In a further aspect, the processing device is further configured to use characteristics of the temporomandibulomaxillary relationship in the design of dental prosthetics for the patient and in diagnosis and treatment of Temporomandibular Disorders (TMD).

In another aspect, the reference object is a color-coded spherical object.

In one aspect, the clamp includes an upper prong, a shaft, and an adjustable lower prong, and wherein the clamp is configured to compress soft tissue surrounding the first bone to hold the clamp in a fixed relationship with the first bone.

In a further aspect, the apparatus further includes a display device connected to the processing device, the display device configured to present a User Interface (UI) that shows images captured by the one or more cameras.

In yet another aspect, the UI is further configured to provide one or more selectable buttons allowing a user to select options for operating the apparatus.

In one aspect, the processing device is configured to utilize Artificial Intelligence (AI) to monitor the characteristics of the movements and positioning of the first bone with respect to the second bone.

In a further aspect, the apparatus further includes one or more of a vibration sensor and an audible capture device for detecting one or more of vibration, sound, and voice at the patient's joint related to movement of the first bone with respect to the second bone to further characterize the patient's joint.

In one aspect, the processing device is further configured to create a homography matrix based on a current image plane with respect to a reference image plane.

In still another aspect, the processing device is further configured to utilize the characteristics of the movements and positioning of the first bone with respect to the second bone to create one or more graphs and contours.

According to one aspect of the present disclosure, a method includes the steps of: arranging a clamp in a fixed position with respect to a first bone of a patient's joint, the clamp having a reference object attached thereto; capturing images of facial landmark points and the reference object; and receiving the captured images and monitoring characteristics of movements and positioning of the first bone of the patient's joint with respect to a second bone of the patient's joint.

In another aspect, the method further includes one or more of the steps of: creating a baseline image including the facial landmark points; and using characteristics of the temporomandibulomaxillary relationship in the design of dental prosthetics for the patient and in diagnosis and treatment of Temporomandibular Disorders (TMD).

In a further aspect, the method further includes the step of presenting a User Interface (UI) that shows the captured images and one or more selectable buttons.

In yet another aspect, the method further includes the step of utilizing Artificial Intelligence (AI) to monitor the characteristics of the movements and positioning of the first bone with respect to the second bone.

In one aspect, the method further includes the step of detecting audible signals at the patient's joint related to movement of the first bone with respect to the second bone to further characterize the patient's joint.

In yet another aspect, the method further includes the step of creating a homography matrix based on a current image plane with respect to a reference image plane.

In one aspect, the method further includes the step of utilizing the characteristics of the movements and positioning of the first bone with respect to the second bone to create one or more graphs and contours.

In another aspect, the audible capture device captures audio recordings of the patient in relation to the temporomandibulomaxillary relationship for baseline reference.

According to a further aspect of the present disclosure, an apparatus is provided that includes at least two cameras arranged to capture images of a head of a patient, a first camera of the at least two cameras arranged toward a front of the head of the patient and a second camera of the at least two cameras toward a side of the head of the patient; and a processing device configured to: receive the captured images from the cameras, detect facial landmarks from the captured images, and monitor characteristics of movements and positioning of the patient based on the detected landmarks.

In one aspect, the processing device is further configured to create a homography matrix based on a current image plane with respect to a reference image plane.

In another aspect, the movements and positioning of the first portion of the patient relative to a second portion of the patient include flexion and extension.

In a further aspect, the apparatus further includes an X-ray sensor diode coupled to the processing device so that when the X-ray energy is landing at the X-ray sensor diode an electrical signals is transmitted to record the patient's positioning degrees during X-rays taken at a particular patient's spatial positioning in space.

In yet another aspect, the first and second cameras are arranged orthogonally to each other.

In one aspect, the apparatus further includes an energy emission device, wherein the processing device is further configured to selectively activate the energy emission device when a position of the head of the patient matches a predetermined position.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 illustrates use of a conventional intraoral fork;

FIG. 2 illustrates use of a conventional electronic facebow;

FIG. 3 illustrates a system for range of motion calculation and positioning in joint imaging in accordance with the present disclosure;

FIGS. 4A-4C illustrate various views of a universal mandibular clamp in accordance with the present disclosure;

FIG. 5 is a flowchart illustrates a method positioning in joint imaging in accordance with the present disclosure;

FIG. 6 illustrates a universal mandibular clamp disposed on a human mandible in accordance with the present disclosure;

FIG. 7A illustrates facial landmark detection of a human head in accordance with the present disclosure;

FIG. 7B illustrates facial lines and screen lines positioned on an image of a face in accordance with an embodiment of the present disclosure;

FIG. 7C illustrates desired positioning of screen lines on an image of a face in accordance with an embodiment of the present disclosure;

FIG. 8 illustrates landmark detection of a side of a human head in accordance with the present disclosure;

FIG. 9 illustrates a screen shot of a Graphic User Interface (GUI) capturing a reference image in accordance with the present disclosure;

FIG. 10 illustrates mapping features in a source image to a destination image in accordance with the present disclosure;

FIG. 11 illustrates a screen shot of detecting and masking a sphere of a universal mandibular clamp in accordance with the present disclosure;

FIG. 12 illustrates mandibular vertical movement and corresponding graph in accordance with the present disclosure;

FIGS. 13A and 13B illustrate graphs of distance vs time for mandibular vertical movement in accordance with the present disclosure;

FIG. 14 illustrates mandibular lateral movement and corresponding graph in accordance with the present disclosure;

FIG. 15 illustrates anterior posterior movement and corresponding graph in accordance with the present disclosure, side camera view;

FIG. 16 illustrates vertical movement and corresponding graph in accordance with the present disclosure, side camera view;

FIGS. 17A and 17B illustrate images for calculating extension and flexion respectively in accordance with the present disclosure;

FIG. 18 illustrates calculation of an angle for flexion and extension in accordance with the present disclosure;

FIGS. 19A-19C illustrate yaw positions of a human head in accordance with the present disclosure; and

FIG. 20 is a block diagram of an exemplary processing device in accordance with an embodiment of the present disclosure.

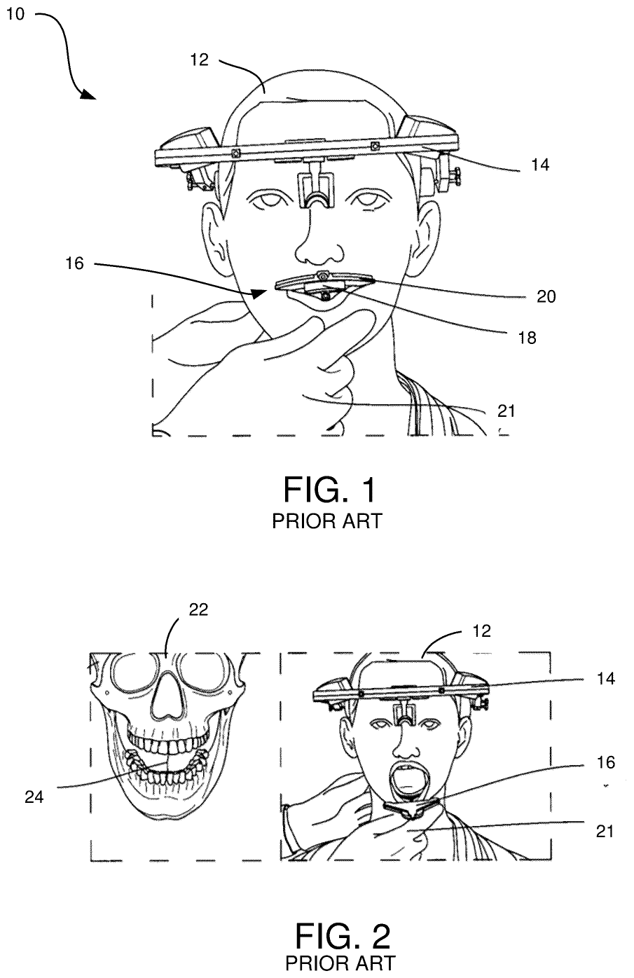

### DETAILED DESCRIPTION

Preferred embodiments of the present disclosure will be described herein below with reference to the accompanying drawings. In the following description, well-known functions or constructions are not described in detail to avoid obscuring the present disclosure in unnecessary detail.

Embodiments of the present disclosure will be described herein below with reference to the accompanying drawings. In the following description, well-known functions or constructions are not described in detail to avoid obscuring the present disclosure in unnecessary detail. The word “exemplary” is used herein to mean “serving as an example, instance, or illustration.” Any configuration or design described herein as “exemplary” is not necessarily to be construed as preferred or advantageous over other configurations or designs. Herein, the phrase “coupled” is defined to mean directly connected to or indirectly connected with through one or more intermediate components. Such intermediate components may include both hardware and software based components.

It is further noted that, unless indicated otherwise, all functions described herein may be performed in either hardware or software, or some combination thereof. In one embodiment, however, the functions are performed by at least one processor, such as a computer or an electronic data processor, digital signal processor or embedded micro-controller, in accordance with code, such as computer program code, software, and/or integrated circuits that are coded to perform such functions, unless indicated otherwise.

It should be appreciated that the present disclosure can be implemented in numerous ways, including as a process, an apparatus, a system, a device, a method, or a computer readable medium such as a computer readable storage medium or a computer network where program instructions are sent over optical or electronic communication links.

In contrast to the conventional sensors, the embodiments of the present disclosure are configured to utilize imaging systems for monitoring Range of Motion (ROM), movement, and positioning characteristics of a patient's teeth, jaws, mouth, etc. The imaging systems do not use typical magnetic sensors or nonsterile reusable equipment. Instead, certain embodiments of the present disclosure are configured to use a simple, disposable clamp that easily attaches to the patient's lower jaw. In particular, the clamp may include a reference object (e.g., ball, sphere) that is used as a reference point for a pair of orthogonal cameras. The cameras can be used with suitable processing devices and/or software to measure characteristics of a patient even if the patient moves his or her head slightly. Therefore, the reference object (or ball) is observable from the perspective of each camera such that adjustments can be made (e.g., using image processing techniques) to the measurements to detect position characteristics, movement, ROM characteristics, and other parameters of a patient's jaw more accurately.

It is to be appreciated that in other embodiments the system of the present disclosure utilizes imaging systems for monitoring Range of Motion (ROM), movement, and positioning characteristics of a patient's teeth, jaws, mouth, etc. without a clamp. In certain embodiments, at least two cameras capture images of a head of a patient and a processing device detects facial feature using an artificial intelligence (AI) function or algorithm to track movement of the head. In other embodiments, at least one camera captures images of a head of a patient and a processing device detects facial feature using an artificial intelligence (AI) function or algorithm to track movement of the head.

The present disclosure provides a predictable, inexpensive, highly accurate and reproducible, interrater reliable processing system (e.g., Artificial Intelligence (AI) system) to study maxillofacial function and TMJs. The techniques of the present disclosure provide the safest methods that do not use any electronic hardware in evaluating range of motion (ROM) and do not utilize any reusable sensors. Instead, the embodiments of the present disclosure use imaging processing techniques from images obtained from the two cameras. These implementations described in the present disclosure allow for excellent reproducibility when reinserted and are very inexpensive and extremely retentive and versatile.

The systems and methods of the present disclosure provide output data to be interrater verifiable and reliable since all steps involved are precisely reproducible. The systems and methods of the present disclosure provide for a precise positioning of a sensor on the mandible, as well, the sensor is a single use device, which makes it very safe, much safer in respect to infectious disease transmission. There is no need to sanitize or autoclave anything since the parts of the apparatus that touch the patient's body are disposable. The systems are configured to track mandibular movements with precision to a fraction of a millimeter.

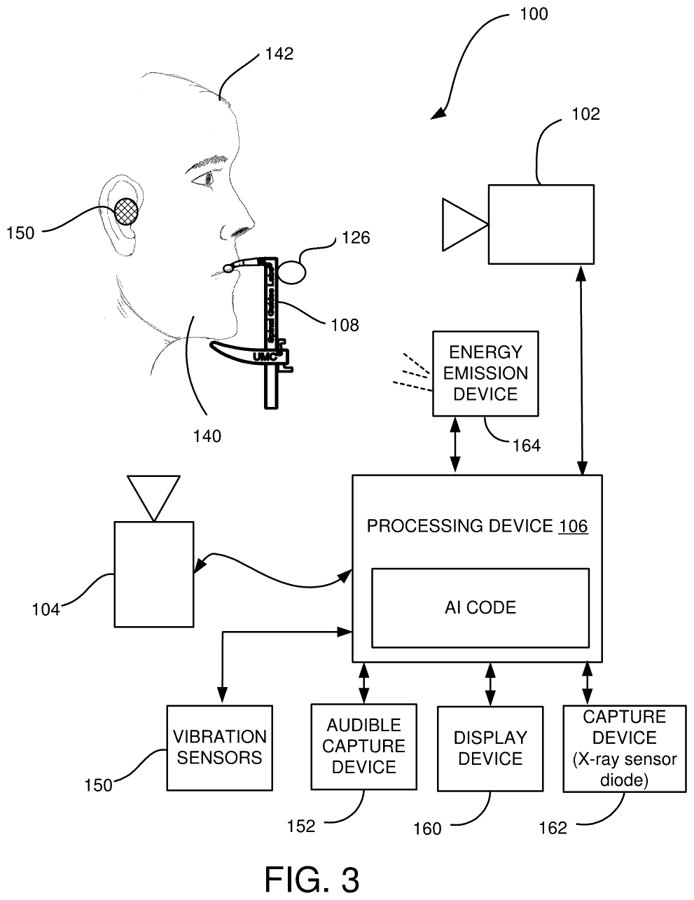

FIG. 3 is a diagram illustrating a system 100 for measuring characteristics of a joint, such as a human jaw. As shown, the system 100 includes a first camera 102, a second camera 104, a processing device 106 (e.g., having AI code or software), a mandibular clamp 108. The mandibular clamp 108 includes at least a reference object 126 (e.g., a color-coded ball), which is used as a reference point for observing positioning and movement of the lower jaw 140 of a patient 142. The first camera 102 may be positioned in the front of the patient 142 and the second camera 104 may be positioned to view the side of the head of the patient 142. The second camera 104 may be positioned at a 90 degree angle relative to the first camera 102. That is, the first and second cameras 102, 104 may be orthogonally positioned with respect to each other. In some embodiments, the first and second cameras 102, 104 may be positioned at about the same height.

As described below, the processing device 106 is configured to receive images captured by the two cameras 102, 104. The cameras 102, 104 are arranged such that captured images include a view of the reference object 126 (e.g., ball). The processing device 106 is configured to calculate a center point (or reference point) representing the center of the reference object 126 and use this center point as a reference that can be compared with points on the face of the patient 142.

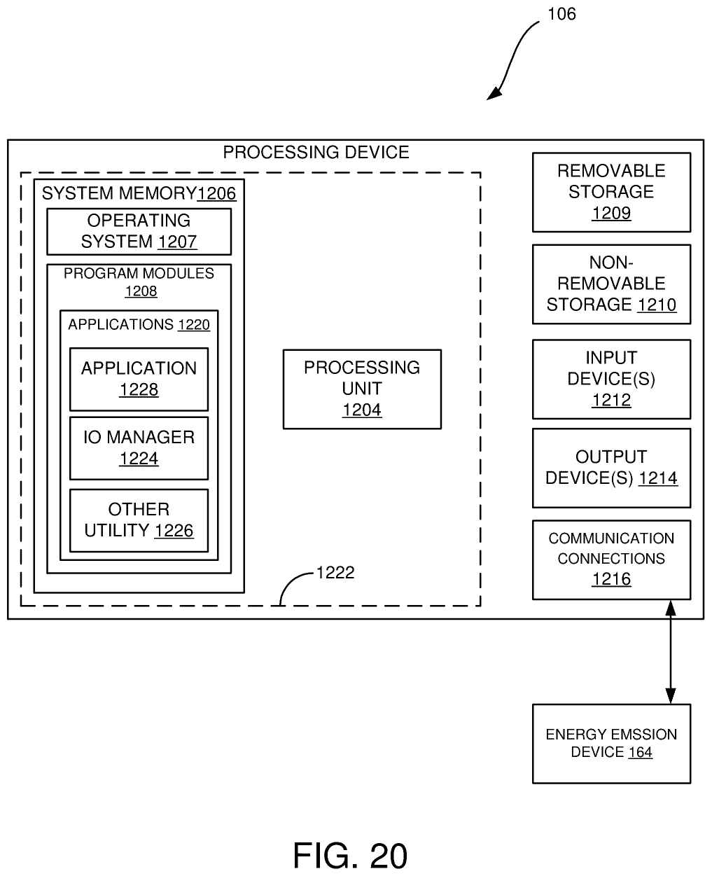

Details of the processing device 106 will be described below in relation to FIG. 20, however, it is to be appreciated that the processing device 106 includes input/output devices such as a display device 106 (e.g., a monitor, touchscreen, etc.), a keyboard, a mouse and at least one speaker. It is further to be appreciated that the processing device 106 may be a single device that includes an imaging device, a display device and input/output devices.

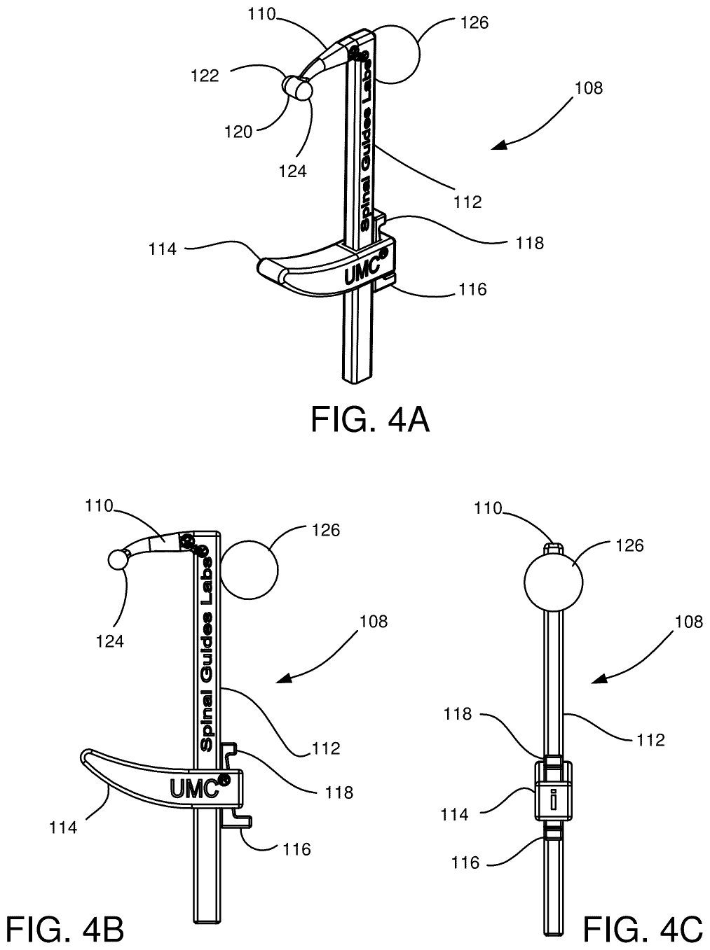

The systems and methods of the present disclosure employ the mandibular clamp 108, essentially for holding the reference object 126 at a fixed location with respect to the face of the patient 142. The mandibular clamp 108, for example, may also be referred to as a Universal Mandibular Clamp (UMC), as shown in FIGS. 4A-4C.

FIGS. 4A-4C show a perspective view, a side view, and a front view, respectively, of an embodiment of the mandibular clamp 108 (e.g., UMC). The mandibular clamp 108, as illustrated, includes an upper prong 110, a shaft 112, a lower prong 114 that is movable with respect to the shaft 112, a wedge lock 116, and a lock release 118. The upper prong 100 may have a “T” shaped head 120 with circular ends 122, 124 on both sides. The head 120 can include any shape but may preferably include the two ball shaped ends 122, 124, as shown, a single cylinder perpendicular to the upper prong 110, or other suitable shape for comfort and an atraumatic experience for the patient 142. Since the clamp 108 may be a single use plastic device, the clamp 108 can be inexpensive and may be safer in terms of infection control among all jaw tracking systems presently available on the market. The clamp 108 may be used for ID by the AI software for the purpose of tracking the mandibular movements and for diagnostic purposes. The center of the reference object 126 can be tracked by the AI software using geometric calculations.

Therefore, no electronic sensors or mechanical sensors are used by the system 100. Since the clamp 108 is lightweight and non-obtrusive, the patient 142 can easily move his or her jaw up and down, side to side, and back to front without obstructions from bulky equipment. Also, the clamp 108 can be applied to the front of the lower jaw in a substantially fixed manner, irrespective of presence of lower teeth, or presence of a deep bite, where the clamp 108 does not move with respect to the lower jaw. No headgear is needed in the system 100. Jaw movements (as well as the speed of these movements) can be monitored and used for creating a patient profile that defines the ROM characteristics, movement characteristics, positioning characteristics, etc. of the patient's jaw (i.e., the temporomandibulomaxillary relationship).

The processing device 106 is configured to use any suitable software, Application Programming Interface (API), hardware, etc. for performing certain monitoring procedures for detecting the positioning and movement characteristics from the captured images. In addition to ROM or positioning characteristics, the processing device 106 may also use a series of images to calculate movement characteristics (e.g., speed) over time.

Also, with the image processing functionality of the system 100, the processing device 106 is configured to compensate for various kinds of head movements, which might be inherent in this field. Various compensation techniques are described below.

Since the system 100 uses substantially exact, repeatable, and reliable data for analysis, the processing device 106 can diagnose prosthetic fabrication solutions for the patient 142 as well as possible issues. This can result in dental prosthetics that are a better fit for the patient 142 compared with conventional systems. In one embodiment, the system 100 may generate a three-dimensional (3D) model of a proposed dental prosthetic, where the model may be provided to a system that constructs the proposed dental prosthetic.

According to additional embodiments, the system 100 may further include vibration sensors 150. In one embodiment, the vibration sensors 150 may be located inside of the external auditory meatus of the patient 142, which is a relatively close place for sensing vibrations coming from TMJ discs anatomically. This location produces the highest sensitivity and lowest noise for vibrations from TMJ discs. It is to be appreciated that the vibration sensors may be located in other positions on the skin surface of patient beside inside of the external auditory meatus. The processing device 106 may receive data from an audible capture device 152, which may correspond with the vibration sensors 150. The processing device 106 may be configured to use phonetics audio recordings in relation to the Temporomandibulomaxillary relationship for baseline reference.

In addition, it may be noted that the system 100 can work in any environment having any magnetic fields since the system 100 does not rely on electronic or magnetic sensing. Thus, the system 100 is impervious to magnetic fields.

In use, the skeletal center of the face of the patient 142 can be determined. The two cameras 102, 104 help in orthogonal positioning of Lateral Flexion and Extension positioning for Cervicocranial Junction (CCJ) or Spinal Imaging for Alteration of Motion Segment Integrity (AOMSI) calculations, which is a problem solving technology.

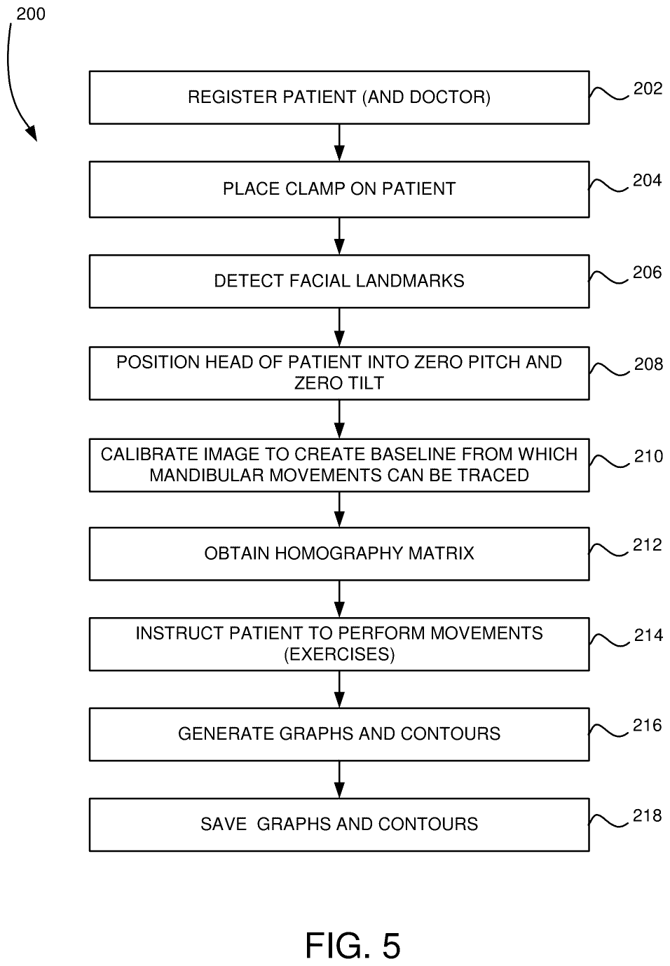

FIG. 5 is a flow diagram showing a process 200 for detecting a patient profile (e.g., ROM, movement, positioning) of a patient's joint, such as the patient's jaw. Specifically, the process 200 may be used for detecting the positioning and movement characteristics of a patient's jaw, such as the temporomandibulomaxillary relationship. In particular, the process 200 does not use electrical and/or mechanical procedures, but instead uses image processing techniques for detecting the patient profile.

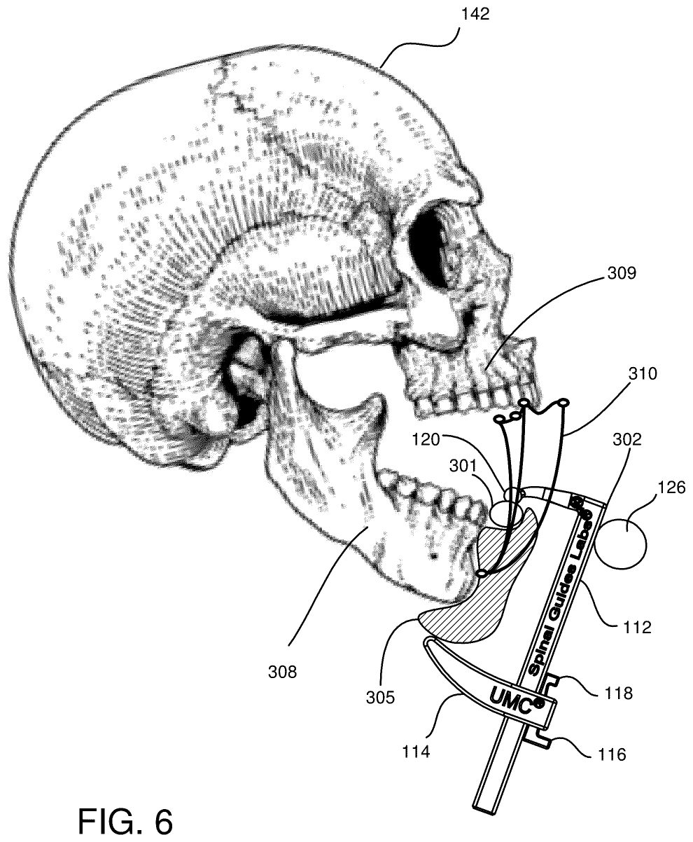

Initially, a patient (and doctor) is registered (step 202). Next, in step 204, the clamp 108 is placed on a patient 142. For example, a skeletal representation of the placement of the clamp 108 on the patient's mandible 308 (lower jaw) is shown in FIG. 6. Sterile gauze 301 (or a cotton roll) can be inserted into the vestibule of the lower jaw for the purpose of providing a cushion and for protecting the soft tissue of the vestibule. The head 120 of the clamp 108 is inserted into the mouth of the patient 142 and placed on the gauze 301. The shaft 112 may be positioned against the soft tissue of chin or may be separated from the chin by a small gap. The lower prong 114 is adjusted (e.g., slid upward on the shaft 112) such that it is positioned against the soft tissue 305 under the chin. The lower prong 114 is configured to be securely and firmly held in place with the wedge lock 116. If the clamp 108 is too tight (or for removal after testing), the lock release 118 may be pressed to achieve a balance between secure attachment to the mandible 308 and comfort for the patient 142.

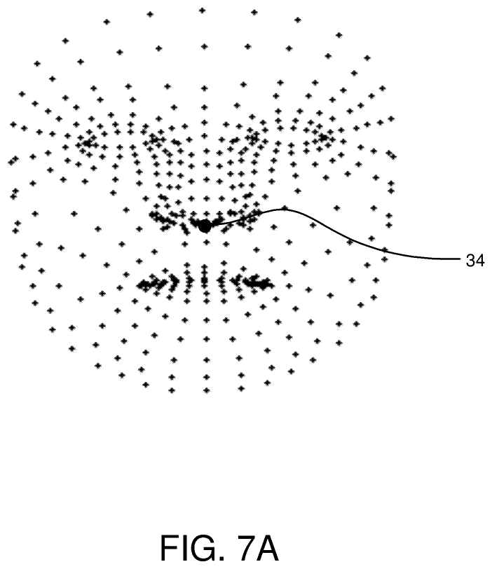

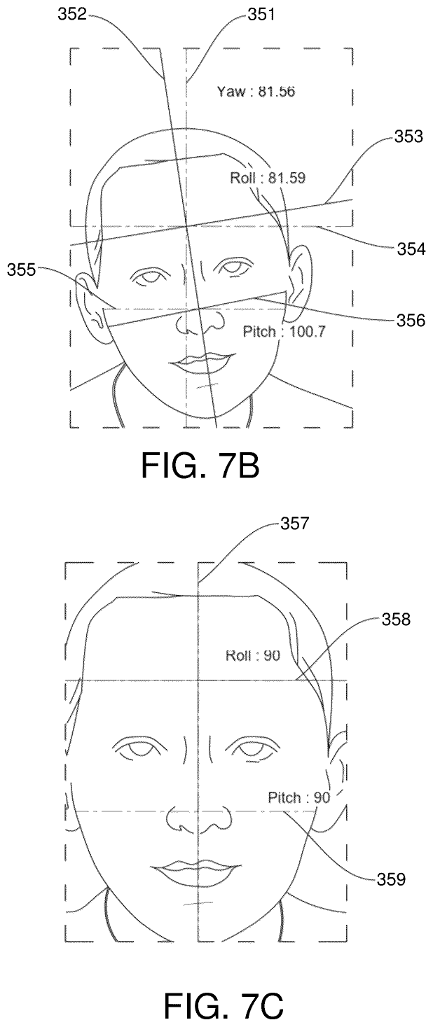

After the step of placing the clamp on the patient (step 204), the process 200 includes detecting facial landmarks (step 206), which is described in more detail with respect to FIG. 7A. Next, with the assistance of a doctor or dentist and/or with instructions from the processing device, the head of the patient is positioned into a zero-pitch and zero-tilt arrangement, as shown in FIGS. 7B and 7C (step 208). A zero-pitch alignment may be achieved by raising or lowering the patient's head and the zero pitch angle is registered when the position of the head is such that Facial Vertical Line 352 and Screen Vertical Line 351 (as shown in FIG. 7B) are moved to create one Single Vertical Line 357 (as shown in FIG. 7C), the Upper Facial Horizontal Line 353 and Upper Screen Horizontal Line 354 (as shown in FIG. 7B) are moved to create one Upper Single Horizontal Line 358 (as shown in FIG. 7C) and the Lower Facial Horizontal Line 356 and Lower Screen Horizontal Line 355 (as shown in FIG. 7B) coincide together into Single Horizontal Line 359 (as shown in FIG. 7C). It is to be appreciated that lines 351, 353 and 356 are generated and drawn based on the detected facial landmarks of step 206, while line 351, 354 and 355 are simply vertical and horizontal lines drawn on the display device 160 to be used to align lines 351, 353 and 356 into a zero pitch angle relative to camera 102.

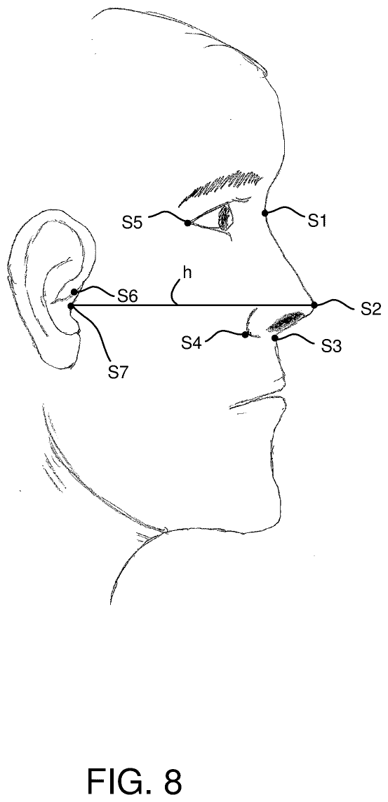

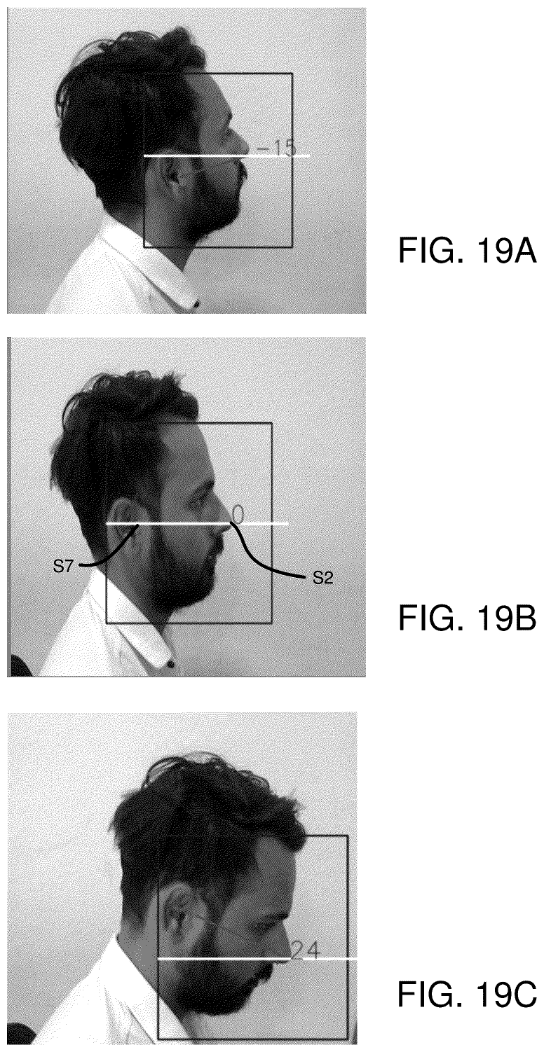

Next, after properly positioning the patient's head, the process 200 includes obtaining a calibration image for creating a baseline Segment S2-S7, as shown in FIG. 8 and in FIG. 19B when Baseline Segment S2-S7 can be viewed on the screen of the side camera from which head movements can be traced for compensation of head movement and from which mandibular movements can then be traced (step 210). A zero-tilt alignment FIG. 19B may be achieved by tilting the patient's head to the left or right so that an angle of the patient's head matches a viewing angle of the first camera 102 directed toward the front of the head.

The process 200 also includes obtaining a homography matrix (step 212) as described in more detail below. Then, according to instructions from the doctor or dentist and/or according to instructions from the AI code, the mandible 308 of the patient 142 is moved in various ways to perform certain exercises (step 214) related to testing the mobility, movement, Range of Motion (ROM), positioning, and other characteristics of the mandible 308, or Temporomandibular Joint. The process 200 further includes generating graphs and contours (step 216) of the patient's joint (e.g., jaw) and saving the graphs and contours (step 218).

Referring back to FIG. 3, when the graphs and contours are generated and saved, this data can be displayed on a display device 160, computer screen, User Interface (UI), Graphical User Interface (GUI), or other suitable display component connected to the processing device 106. Thus, the data can be presented to a doctor or dentist to show the captured images, instruct the doctor or patient with respect to movements or exercises for testing the ROM or other characteristics of the jaw, and show data, results, patient profiles, etc. of the monitored movements.

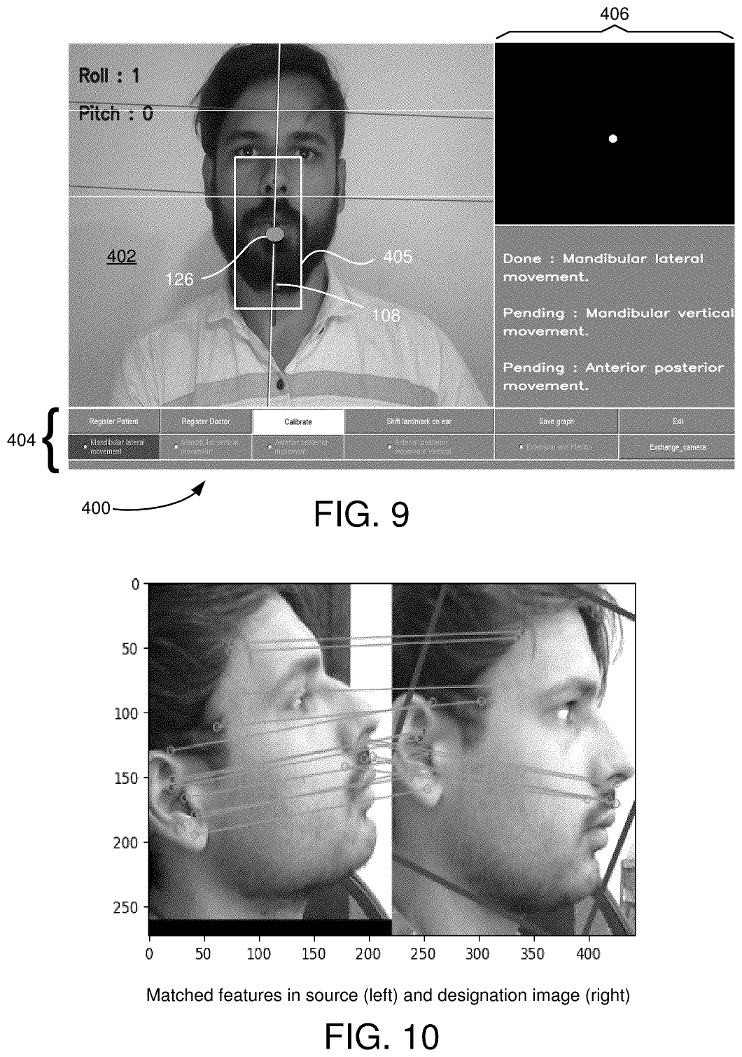

The system 100 can find markings of a skeletal center, which can be illustrated on the display device 160. The skeletal center can be shown as an image captured by the first camera 102 made during skeletal center identification with the help of the AI software. The skeletal center relates to the facial vertical line (e.g., generated by the AI software) when it meets the computer vertical line (e.g., generated as the center line of the display device 160) and/or when a Single Vertical Line is established. These various vertical lines are shown in the images on the display device 160 in FIGS. 9, 11, 12, 13A, and 14. The Single Vertical line corresponds to the Skeletal Center. The clamp 108 can be attached to the mouth such that the clamp 108 and the reference object 126 are aligned with the Skeletal Center and the lower prong 114 is located below the chin tightly locked in with the wedge lock 116. Intraoral procedures can be performed with or without local or topical anesthetics, which may be applied for the patient's comfort. Locking of the clamp 108 is attained via applying firm pressure onto the wedge lock 116. A lateral clamp point 302 (FIG. 6) can be used as a reference point in addition to the reference object 126 (e.g., color-coded ball). Observing the movement of the reference object 126 with respect to fixed points on the patient, the processing device 106 (e.g., using hardware, software, firmware, AI code, etc.) is configured to detect the positioning of the mandible 308 with respect to the maxilla 309 (i.e., upper jaw) at all times during the test.

FIG. 7A is a computer-generated image obtained by observing the facial landmarks (FIG. 5, step 206). For example, a first landmark detection step by the first camera 102 can be obtained of the front or face, which is illustrated. These landmark points may be used to obtain orientation information. Additional points may also be added using the second camera 104. In one embodiment, the landmarks may be detected by a pretrained AI model (e.g., mediapipe from Google API). This model may be configured to detect up to a certain number of points on the face of the patient (e.g., 468 landmark points). In one embodiment, a tip of the nose can be identified as point 34.

Any kind of movements (e.g., opening one's mouth, closing one's mouth, chewing, swallowing, etc.) can be traced. Also, using a sequence of movement images over time, speed and/or acceleration parameters can also be calculated to determine other parameters of the joint movement. The movements can result in changes to the landmark points and can also be correlated with the position of the reference object 126. Based on the position of the reference object 126 with respect to the landmark points from the point of view of both the first and second cameras 102, 104, the processing device 106 can detect the joint movement characteristics (e.g., using AI).

Camera 104 from the side of the head can estimate the movement in the Cervicocranium and Neck. The second camera 104 can be used for detecting at least the following reference points, as shown in FIG. 8:
Upper Nose Point S1
Tip of the Nose S2
Point S3 at the angle between nose and upper lip
Point S4 at the angle between the alar of the nose and cheek
Point S5 at the corner of the eye
Point S6 at the angle between tragus of the ear and the cheek
Point S7 at the Horizontal line h drawn from S2 Point (Point 34 shown in FIG. 7A) going back and placed at the intersection with the border of the ear model at the Pitch Zero.

The reference points and other dimensions are used in orthogonal Neutral, Flexion and Extension of the Neck positioning for diagnostic imaging utilizing mentioned points and horizontal line (h). The horizontal line h can be drawn from the points S2 (or point 34 shown in FIG. 7A) at the moment of step 210 and is extended backwards. The point S7 stays on the Horizontal drawn from point S2 (Point 34) going back and placed at the intersection with the border of the ear model at the Pitch Zero. Segment S2-S7 is adjusted as Neutral at the moment when Segment S2-S7 coincides with horizontal line h at the moment when the head is positioned at the Zero Pitch with the first camera 102 when the yaw and roll are at 0 degrees and the Vertical Center of the Face and Vertical Screen Center of the focus of the camera 102 coincide.

In certain embodiments, the head positioning system 100 that includes both cameras 102, 104 and processing unit 106 can be used for radiation oncology treatment and proton radiotherapy on head and neck. In one embodiment, both cameras are orthogonally positioned towards the head of the patient, i.e., one camera facing the front of the patient's head while the second camera faces the side of the patient's head, and the head can be positioned and precisely repositioned in space without the use of UMC clamp, audible capture device and vibration censors utilizing aforementioned lines and facial points. In other embodiment, one camera may be used to monitor, track, position and precisely reposition the head of the patient in space without the use of the UMC clamp.

Once the head is positioned at Pitch Zero with the front camera 102 and step 210 initiating the steps involved with the side camera 104, the processing unit 106 creates the horizontal line h to extend at the Point S2. The point S7 stays on the Horizontal drawn from point S2 (Point 34) going back and placed at the intersection with the border of the ear model at the Pitch Zero creating the Segment S2-S7, see FIG. 8.

The processing device 106 records precise geometrical coordinates of facial landmarks points from the side camera 104 in relation to the horizontal line h and from the front camera 102 in relation at Pitch Zero at the moment of initial diagnostic CT scan. This provides hard coordinates for precise and controlled positioning of the head and neck in space relative to both cameras.

Since both cameras and therapeutic radiation oncology equipment can be fixed and stationary, the patient's head and neck can be moved in space within the field of the two cameras 102, 104 to the desired position. The patient's head and neck can be positioned either by the patient, or with use of special robotic tables into predetermined coordinates at the diagnostic imaging appointment coordinates. This enables the operator to position patient's head within given coordinates in relation to both cameras and reposition the head as many times as necessary with precision without constant X-ray exposures for monitoring of imaging guides. Both cameras provide data for monitoring and tracking for precise head positioning. This method can be used in the system for precise human head positioning within given coordinates utilizing AI as an “ON” and “OFF” switch within the space between the two orthogonally oriented cameras 102, 104 while conducting radiation oncology procedures, or proton therapy on head and neck. For example, a precisely positioned head triggers the processing device 106 to send an “ON” signal to an energy emission device 164, e.g., an ionizing high energy generating device, and emission starts. It is to be appreciated that exemplary ionizing high energy generating devices may include, but is not limited to, a high energy photon generating device, a X-ray device, a gamma ray device, etc. A minute deviation of head positioning results in the processing device 106 triggering an “OFF” signal to the energy emission device 164 to stop radiation robotic surgery treatment.

Currently, the state of art for precise positioning for radiation oncology/robotic surgery is obtained under guidance and correction with the use of X-ray imaging of radiological guides while monitoring positioning to make sure the precise position obtained within the presurgical diagnostic coordinates obtained by diagnostic CT scan. Irradiation, proton therapy started once the processing unit that processes the radiological guides determines that the head is precisely positioned within known predetermined positioning in space obtained by initial diagnostic CT with the guides. A minute deviation in positioning results in the processing device 106 triggering an “OFF” switch to interrupt the treatment. Head positioning is a challenge since even while breathing the head is moving necessitating the patients to hold breathing while in treatment which is a very difficult task because even then when the patient is not breathing the head can be moving. Minute movement of the head while the treatment is “ON” causes serious damage to surrounding tissues crippling, even killing patients. This equipment is extremely expensive, and while in use, additionally exposes patients to high amounts of harmful X-ray radiation due to constant x-ray control.

Utilizing the head positioning system with two video cameras as described is simple, inexpensive, is much safer due to reduction of X-ray radiation exposure, provides real time positioning controls with higher precision and a selective “ON” and “OFF” function depending on precision of head positioning without constant lengthy X-ray radiation exposures. Monitoring of positioning is constant, in real time, through video cameras and without any X-ray radiation. It is to be appreciated that the selective “ON”/“OFF” control of the high energy emission device may be performed using one camera by detecting using an AI function to detect facial features and track the detected features for positioning and repositioning. Current technology of head and neck positioning for the purposes of radiation robotic treatments does not provide real time positioning and still remains a serious challenge and a problem area in medicine.

Referring again to step 208 of the process 200 of FIG. 5, the detected landmarks are employed to position the head of the patient into zero pitch. An Upper Face Horizontal Line (e.g., such as line h, generated by the AI software) coincides with an Upper Computer Horizontal Line to form Upper Single Horizontal Line. The Vertical Computer Line coincides with Vertical Facial Line to form Vertical Single Line (e.g., Skeletal Center). The Lower Horizontal Line equals and coincides with a Pitch Line.

The system 100 identifies a Relativity Point (e.g., point 34 on the tip of the nose), which can be any other reference point on the face (e.g., upper ⅔ of the face) and does not move while the clamp 108 translates with the Mandibular movement. All further openings and closings of the Mandible, as well as other movements, can be referenced from that Relativity Point and the Center of the reference object 126 on the clamp 108.

The AI software can use the reference object 126 to detect points relative to an unseen central point within the reference object 126. The system 100 identifies the center of the reference object 126 and from that center all calculations can be made. In some embodiments, the reference object 126 can have a spherical shape, which can help for identifying a specific center point, whereas other types of shapes may be distorted when recognized by the software from the captured images since calculations from distorted center points may be not as precise and reliable as in the case with the center from a spherical shaped optical tracker. The reference object 126 can be perceived by the software as a ball from all and any angle without distortion because it is using direct optics, and in comparison, for example with an indirect optics as can be seen from an X-ray image of a ball where it is very important to have as close to 90 degree X-ray source to object angulation as possible. Further away from the ball to a side, more distortion may be experienced, and the shape of a ball may appear as an oval. In direct optics, however, the appearance is the same. That is, a sphere will always appear as a sphere. On the other hand, a square from different angles will have different apparent outlines and can look different from different angles and therefore is not optimal as a reference object for tracking its center with direct optics of AI software.

FIG. 9 is a screenshot showing a UI 400 that may be presented on the display device 160. As shown in this embodiment, the UI 400 may include a camera view 402 that may show in real time the image captured by the first camera 102. In some cases, a user may select the side view to show the images captured by the second camera 104 directed toward the side of the patient's head. In addition, the UI 400 includes a number of buttons 404, options, selections, etc. that the user may select. For example, with respect to step 202 of the process 200 of FIG. 5, the user may select one or more of the buttons 404 to register the patient and/or the doctor.

Also, the buttons 404 may include a “calibrate” button that allows the user to initiate a calibration step in which the processing device 106 calibrates captured images with computer-generated horizontal or vertical lines. The calibrate button may correspond with step 210 of the process 200, where an image is calibrated to create a baseline from which mandibular movements can be traced. To compensate for the movement of the head, a reference image is obtained by clicking on the calibrate button. Once this button is clicked, the processing device 106 is configured to use a homography process where the center of the clamp 108 is mapped to compensate for movement of the head. This can be obtained by calculating the center of a mask 405, which can be displayed in the camera view 402 in a reference plane. The homography process, for example, may include projecting or transforming one image plane to another. In other words, the homography process defines the relationship between two images when the patient's head moves. Thus, homography can be used to compensate for the movement of the head and is configured to map points on a currently captured image plane to a reference plane, according to step 212. The reference plane, for example, may be the initial image that is captured when the system is first calibrated. In some cases, the horizontal and vertical lines representing facial landmark points may be aligned with the horizontal and vertical reference lines of the cameras 102, 104 to calibrate the system. For example, when the pitch lines are matched with respect to the perspective of the second camera 104 and the tilt lines are matched with respect to the perspective of the first camera 102, the reference plane can be obtained. From this reference plane, new images can be compared.

Again, homography can be described as a mapping between two projective planes with the same center of projection. To obtain a homography matrix (step 212), corresponding features (e.g., at least 4) in the source and destination images may be used. The processing device 106 is then configured to map the center point of the clamp 108 to the reference plane. As shown in FIG. 10, the first example of a patient shows the patient with his head pitched back. Then, with proper alignment instructions (e.g., from the doctor or AI software), the patient can be asked (or can be assisted) to “lower your chin” (or other instructions) to align the horizontal lines with the reference.

The relation between the source plane and the destination plane is given by following equations:

$$( \begin{matrix}{WX}_{1} & {WX}_{2} & {WX}_{3} & {WX}_{4} & & {WX}_{n} \\ {WY}_{1} & {WY}_{2} & {WY}_{3} & {WY}_{4} & …… & {WY}_{n} \\ W & W & W & W & & W\end{matrix} ) = ( \begin{matrix}{h}_{11} & {h}_{12} & {h}_{13} \\ {h}_{21} & {h}_{22} & {h}_{23} \\ {h}_{31} & {h}_{32} & {h}_{33}\end{matrix} ) ( \begin{matrix}{x}_{1} & {x}_{2} & {x}_{3} & {x}_{4} & & {x}_{n} \\ {y}_{1} & {y}_{2} & {y}_{3} & {y}_{4} & …… & {y}_{n} \\ 1 & 1 & 1 & 1 & & 1\end{matrix} )$$

where X₁, Y₁ and x₁, y₁ are coordinates of corresponding features in source and destination images. The corresponding features are obtained using landmark detection and image processing descriptors (e.g., SIFT descriptors) that may include features that can be used to obtain landmarks. These techniques may be used for detecting salient, stable feature points in an image. For every such point, it also provides a set of “features” that “characterize/describe” a small image region around a point. These features are invariant to rotation and scale. For example, in some cases, Random Sample Consensus (RANSAC) algorithms may then be used to remove outliers. RANSAC is a resampling technique that generates candidate solutions by using the minimum number observations (data points) required to estimate the underlying model parameters.

Once the source image has been mapped to the reference plane, the processing device 106 is configured to map the center point of the reference object 126 of the clamp 108 to the reference image (at pitch 0). In this way, the processing device 106 is able to compensate for the movement of the head. If the coordinates for the center of the clamp 108 is (x_{c}, y_{c}) in current plane, for example, then its coordinate in reference plane (X_{R}, Y_{R}) can be obtained by following equation:

$$( \begin{matrix}{WX}_{R} \\ {WY}_{R} \\ W\end{matrix} ) = ( \begin{matrix}{h}_{11} & {h}_{12} & {h}_{13} \\ {h}_{21} & {h}_{22} & {h}_{23} \\ {h}_{31} & {h}_{32} & {h}_{33}\end{matrix} ) ( \begin{matrix}{x}_{c} \\ {y}_{c} \\ 1\end{matrix} )$$

The system of direct optic recognition works when different shades are identified by a learned system. Direct optics is an undistorted view of a 3D object, as one can see with his own eyes. Indirect optics is an image of a shadow of an object, which makes an observer deduce the original shape of the object by knowledge of how it looks in reality. According to one example, a doctor knows how the bone anatomy looks and from the shadow of the X-ray can deduce a fracture. Different shades may appear as the spectrum may change due to different reflections, or different light spectrum arriving at an object to be identified and then the reflection of it creates different noises, and the software picks up those noise events and shows it as different targets mixing them with the one it needs. In one embodiment, the reference object 126 may be a sphere, may have a green color, and may have a size of about 15 mm in diameter.

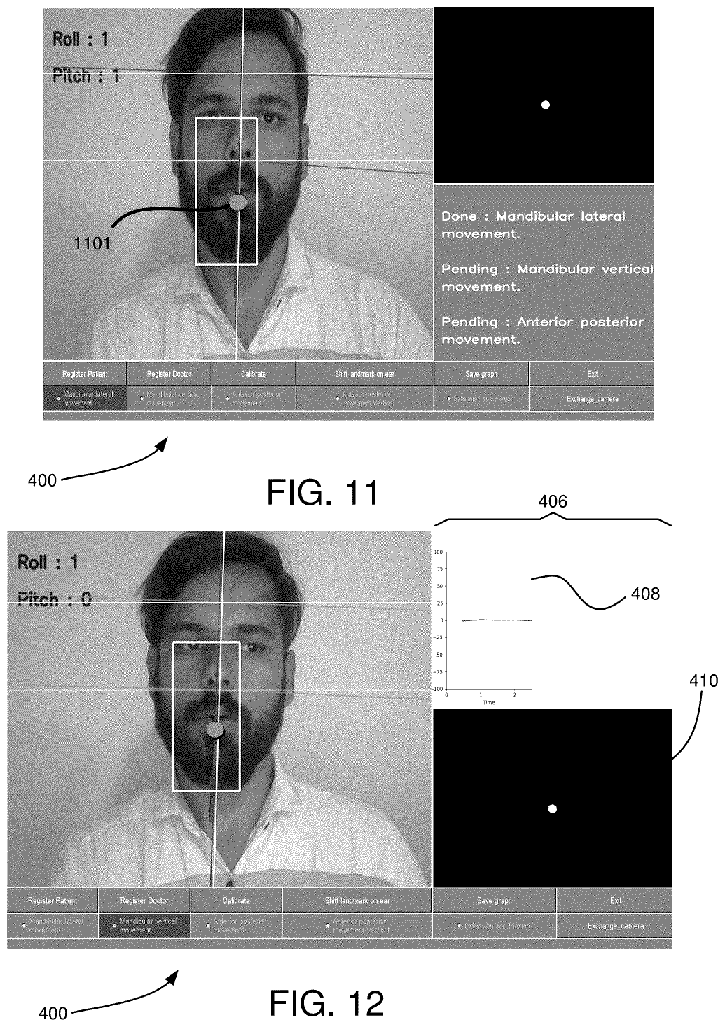

To detect the green color of sphere 126, the image is converted to Hue, Saturation, Value (HSV) format. The Hue specifies the angle of the color on the RGB color circle. A 0° hue results in red, 120° results in blue, and 240° results in green. Saturation controls the amount of color used. A color with 100% saturation will be the purest color possible, while 0% saturation yields grayscale. The Value controls the brightness of the color. A color with 0% brightness is pure black while a color with 100% brightness has no black mixed into the color. Because this dimension is often referred to as brightness, the HSV color model is sometimes called HSB, including in P5.js. The processing device 106 can extract only those pixels which are in the range from [45,50,50] to [75,255,255]. The pixels are extracted in the form of a mask. If the pixels values for HSV color-codes are in this range, then those pixels are masked 1101, where the pixels can have value 0 or 1, as shown in FIG. 11.

It may be noted that the cameras 102, 104 can work simultaneously. In some embodiments, the first camera 102 can be used for continually obtaining the front image. The second camera 104 from the side can work when the side points are needed for recognizing pitch alignment.

After the homography matrix is generated (step 212), additional movements can be tracked. A homography can be a transformation that maps points from one plane to another. In computer vision, it can often be used to relate points in one image to corresponding points in another image taken from a different viewpoint. In this case, the reference image and the current image are likely taken from slightly different perspectives due to the movement of the head. After generating the homography between the reference image and the current image, appropriate compensation can be made. Then, the center point of the clamp is mapped on the reference image.

In step 214, the patient will be instructed to perform various exercises as will be described below. From the exercises, graphs and contours will be generated (step 216) and the generated graphs and contours may be saved (step 218).

For example, after clicking on the Calibrate button and selecting the “Mandibular vertical movement” button, a graph and a contour map can be shown at the right side of the UI 400, which corresponds to a test results section 406, as shown in FIG. 12. The graph 408 shows the relationship between distance and time. Distance is calculated between reference position and current position of center of clamp. Initially, when the mouth is stationary, it may be noted that the graph 408 is substantially a flat line and a contour map 410 shows only a dot. When observations are completed, the user can click on a “save graph” button to save the graph. After that, the UI 400 provides two options, where the first is for capturing a “Rest Position” and the second is for capturing “Maximum opening and closing” positions. Since mouth is not moving in this example, the user may select the option of the “Rest Position.”

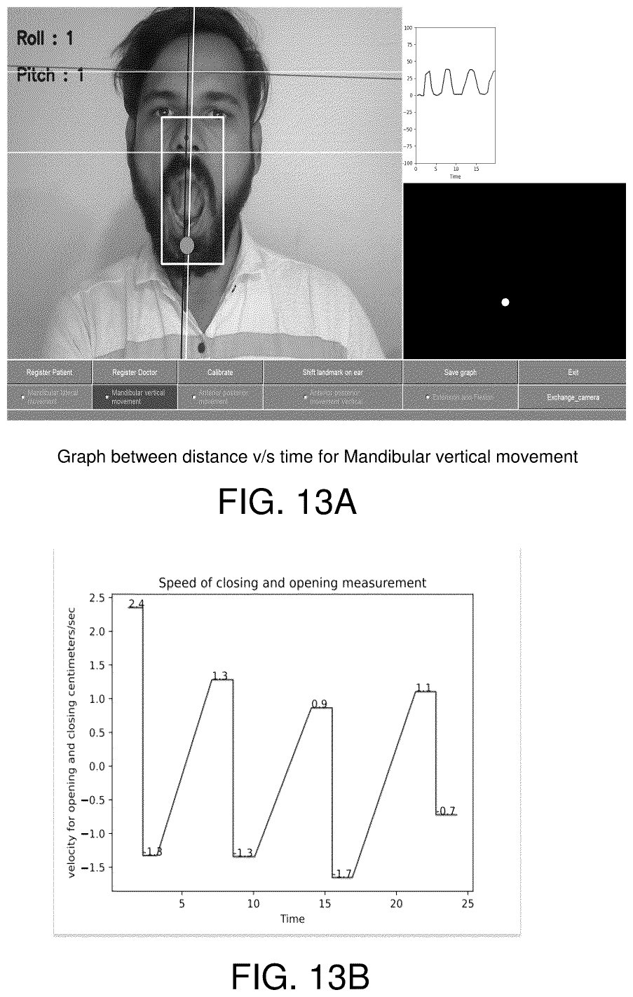

The exercise related to the “Maximum opening and closing” option for “Mandibular vertical movement,” the system 100 shows that the user can perform the exercise for “Maximum opening and closing” for “Mandibular vertical movement,” as shown in FIG. 13A. Here, the contour is a vertical line and the graph for distance and time is in the top right corner, as shown in FIG. 13A.

FIG. 13B shows vertical lines as opening and closing and short horizontal lines pause between opening and closing within timing coordinates and travel. The significance is that when there is pain, the graph is different because either/or opening and/or closing becomes slower and smaller in amplitude in contrast with normal values. This may be a graph of speed vs time for opening and closing.

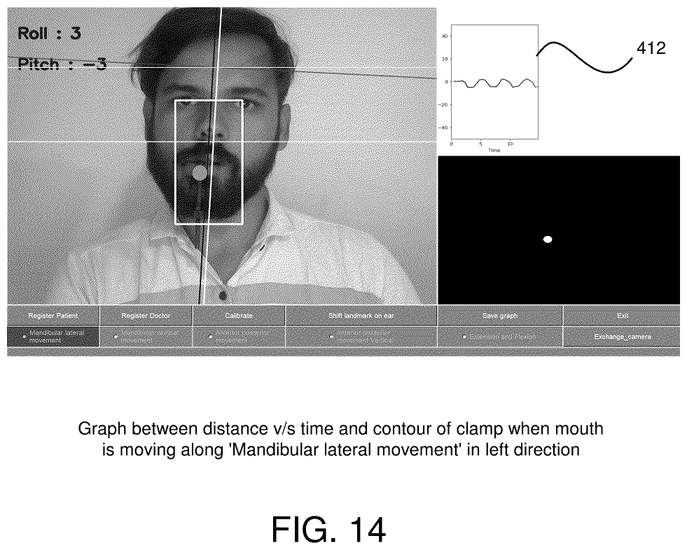

As shown in FIG. 14, the UI 400 shows that the patient is performing exercises for “Mandibular lateral movement” in one direction (e.g., lateral movement to left from the perspective of the first camera 102). Here, the distance and time graph 412 has been shown in the top right corner of the UI 400 where distance is measured negatively since movement is in left direction or in negative x direction. When observations are completed, the user (e.g., doctor, technician, medical assistant, etc.) can click on the ‘Save graph’ button to save the graph. After that, the UI 400 provides two options: ‘Mandibular lateral movement left side’ and ‘Mandibular lateral movement right side’.

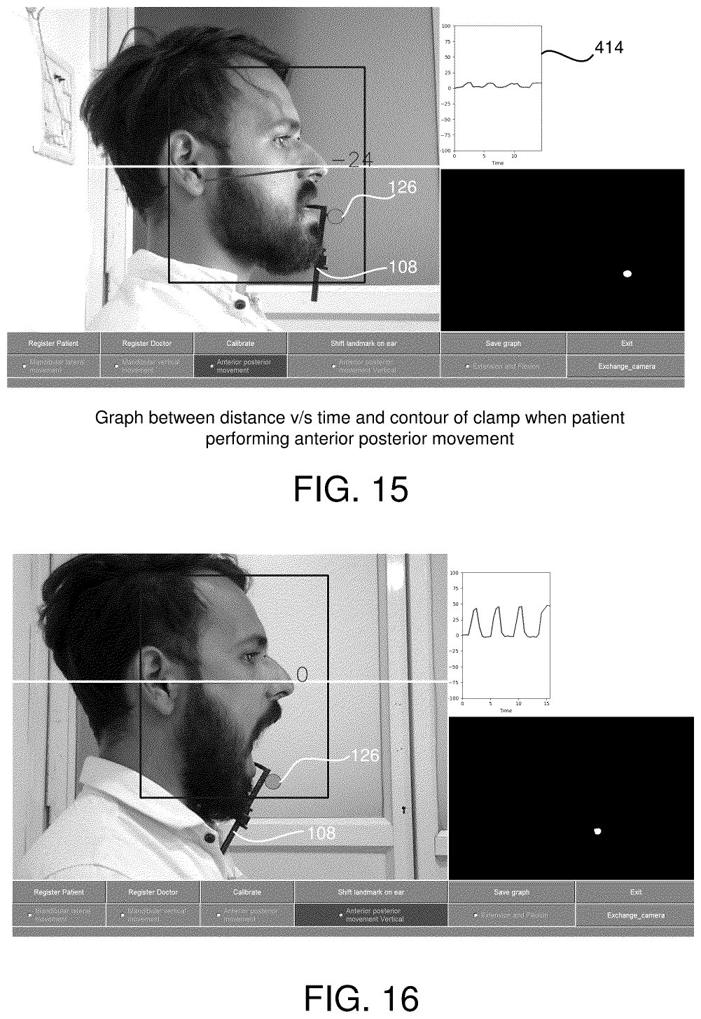

As shown in FIG. 15, the patient is allowed to perform an exercise of Anterior Posterior movement. The patient performs this exercise and images again are captured. The contour graph 414 in this case is a horizontal line but the contour length may be very small along the z axis. The contour graph 414 for distance and time can be displayed in the top right corner of the UI 400. For example, the contour line is the sphere's travel trajectory from the side camera view traced as a horizontal line (e.g., jaw forward—backward movement) and may be small.

FIG. 16 shows the patient performing another exercise. In this example, the patient performs a maximum opening and closing action to obtain a Lateral View.

Flexion and extension actions describe movements that affect the angle between two parts of the body. Flexion describes a bending movement that decreases the angle between a segment and its proximal segment. Extension is the opposite of flexion, describing a straightening movement that increases the angle between body parts.

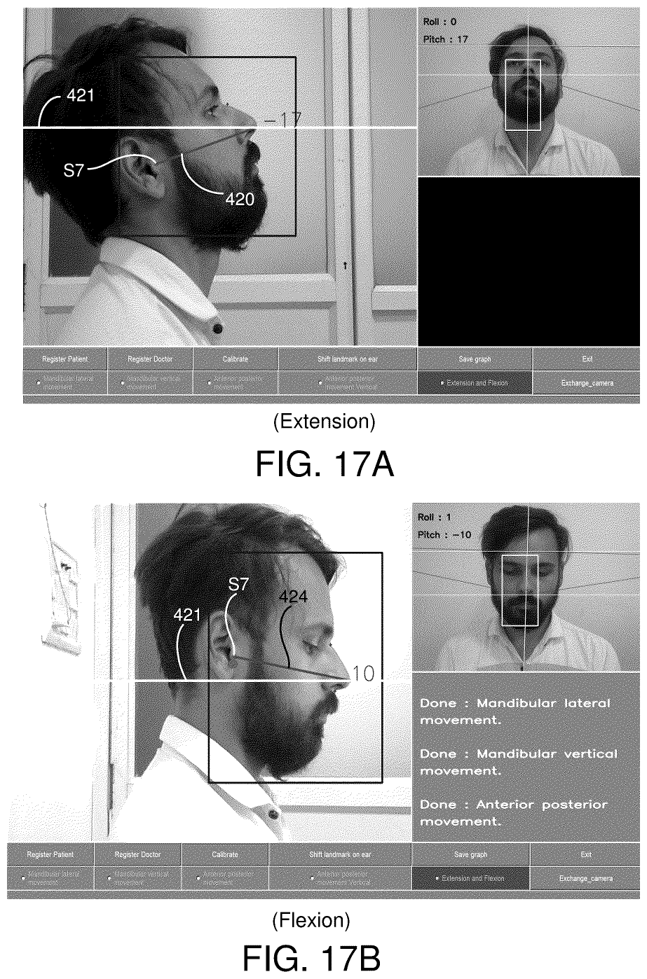

FIGS. 17A and 17B show the calculation of extension actions and flexion actions respectively. The UI 400 shows the side view of the patient from the perspective of the second camera 104. Also, the front views are shown in the supplemental boxes in the top right corner of the UI 400. The user can select the button labelled Shift to shift the landmark on the ear to obtain the reference line where the patient is along the horizontal line. Here, the processing device 106 can again use homography to obtain the shifted landmarks. All the calculations may be the same as discussed above. For example, a point along the horizontal line just above the landmark is detected on the ear but passes through the horizontal line that in turn passes through the landmark point detected on the nose. To detect this point, the processing device 106 allows the user to press the “Shift landmark on ear” button when the pitch (e.g., head tilt forward or backward) and roll (e.g., head tilt left or right) are zero and the yaw (e.g., head swivel side to side) is neutral (or not applicable) for the front camera view. In one embodiment, this arrangement includes the use of both cameras 102, 104 arranged orthogonally with respect to each other and may be essentially at the same height. It is to be appreciated that in other embodiments, the cameras are not required to be arranged orthogonally with respect to each nor at the same height. In other embodiments, camera 102 may be arranged at an adjustable angle relative to camera 104.

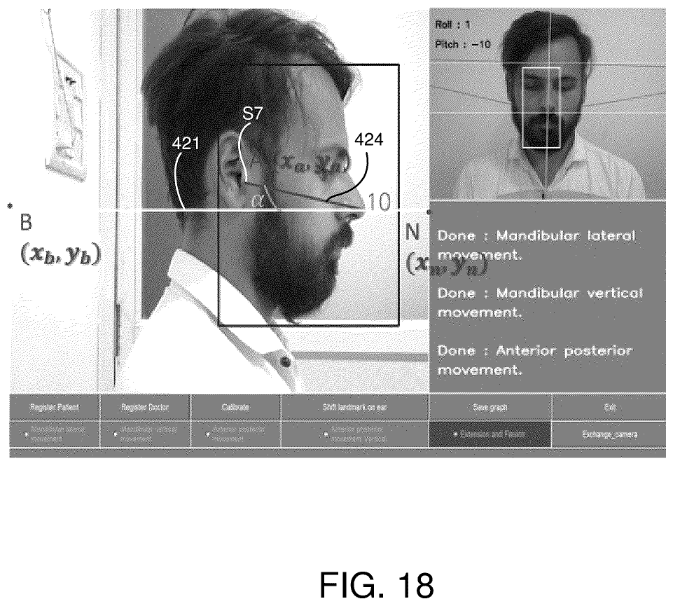

As shown in FIG. 17A, the patient has his head tilted backward, e.g., performing an extension action. In this example, the S2-to-S7 Baseline 420 is angled (e.g., pitch=17 degrees) with respect to the camera's horizontal line 421. To register a pitch line 424 is drawn (e.g., pitch=−10 degrees) and S7 is a dynamic point. The angle between the computer horizontal line 421 and the S2-to-S7 line 424 determines the flexion and extension angle (e.g., angle α as shown in FIG. 18, which will be described below), along with the pitch angle from the front camera. A rotation matrix can be used to obtain this point. Points on FIGS. 17A and 17B may correspond to the S7 point.

In this example, the second camera 104 is used for homographic estimation of head movement during flexion and extension while the mandible is opened and closed when various jaw movements are performed so that the movements of head are compensated while opening and closing is done. For mandibular movement to check head movement in Cervicocranium, the clamp 108 is positioned in the mouth. When the cervical spine is studied for orthogonal images, some embodiments do not need the mandibular clamp.

Furthermore, for clarification, the front side camera can be used for detecting movement about the y-axis (opening and closing of the jaw, deviation, and deflection of the jaw) and for detecting movement about the x-axis (lateral movement to the left and right sides). The side camera can be used for detecting movement about the z-axis (jaw going in protrusive and retrusive movements).

According to some embodiments, the front camera may always be working, and the side camera may be added when side points are needed to be checked (e.g., when z-axis, flexion, and extension movements of the neck are to be checked). If a point (e.g., S7 point in FIG. 8) is a selected point in a reference image, which is along the horizontal line, then this point should move along the face also so that the processing device 106 can measure the angle of flexion and extension. For this, the processing device 106 can calculate the homographic matrix between the reference plane and current plane. Then, the coordinate points, which are selected along the horizontal line in the current frame, will be:

$$( \begin{matrix}{WX}_{c} \\ {WY}_{c} \\ W\end{matrix} ) = ( \begin{matrix}{h}_{11} & {h}_{12} & {h}_{13} \\ {h}_{21} & {h}_{22} & {h}_{23} \\ {h}_{31} & {h}_{32} & {h}_{33}\end{matrix} ) ( \begin{matrix}{x}_{r} \\ {y}_{r} \\ 1\end{matrix} )$$

If coordinate of point N and A are (xₙ,yₙ) and (xₐ,yₐ), respectively, then the slope of AN is:

$$M = Slope of AN = \frac{{y}_{n} - {y}_{a}}{{x}_{n} - {x}_{a}}$$

Similarly, the slope of NB is:

$$N = Slope of NB = \frac{{y}_{n} - {y}_{b}}{{x}_{n} - {x}_{b}}$$

The angle for flexion and extension a is calculated as below (see FIG. 18):

$$α = \{ \begin{matrix}+ {tan}^{- 1} ( \frac{\vert M - N \vert}{i - MN} ) , & if M > N \\ - {tan}^{- 1} ( \frac{\vert M - N \vert}{1 + MN} ) , & if M < N\end{matrix}$$

In FIG. 18, angle α is determined for a flexion action. In a similar manner, the angle α for an extension action may be determined between lines 420 and 421 as shown in FIG. 17A.

When the test performs movement for extension and flexion, the patient's face should be along the neutral yaw position. This may be done to ensure that rigid body transformation is used. Since it is known that the face is a rigid body, if the patient is moving his or her jaw (e.g., the distances between each key point, such as nose, ear, mouth, eye, etc. is constant). But in front of the camera, if we are at constant distance from it and we are not in neutral yaw position we can see in FIGS. 19A and 19B that distance between key points does not appear to be same as it was in neutral yaw position. In FIGS. 19B and 19C, it can be seen that face is in neutral yaw position and not rotated.

It is clear that in neutral yaw position, rigidness is maintained in camera frames for face key points. To observe the rigidness of facial key points we use rigid transformation for rotation.

Firstly, a reference frame is taken in which all the facial key points are stored. In this reference frame, the patient should be in neutral yaw position and extension and flexion should also be zero. Then, the rigid transformation is calculated for rotation in between key points of reference frame and key points of real time frame. If we have (X₁, Y₁), (x₁, y1), (X₂, Y₂), (x₂, Y₂) . . . . (Xₙ, Yₙ), (xₙ, yₙ) are corresponding points in reference frame and real time frame, then relation between these corresponding points and rotation matrix is as follows:

$$( \begin{matrix}{X}_{1} & {X}_{2} & {X}_{3} & {X}_{4} & & {X}_{n} \\ {Y}_{1} & {Y}_{2} & {Y}_{3} & {Y}_{4} & …… & {Y}_{n} \\ 1 & 1 & 1 & 1 & & 1\end{matrix} ) = ( \begin{matrix}cos θ & sin θ & 0 \\ - sin θ & cos θ & 0 \\ 0 & 0 & 1\end{matrix} ) ( \begin{matrix}{x}_{1} & {x}_{2} & {x}_{3} & {x}_{4} & & {x}_{n} \\ {y}_{1} & {y}_{2} & {y}_{3} & {y}_{4} & …… & {y}_{n} \\ 1 & 1 & 1 & 1 & & 1\end{matrix} )$$

$$or A = R * B$$

Calculation of rotation matrix R is done using singular value decomposition. Any matrix A can be factorized in three matrices such that:

M=USV^{T}

If A is a point set in reference plane and B is a point set in a real time frame, then their centroids are:

$$\begin{matrix}{C}_{A} = \frac{1}{η} {\sum }_{i} {A}_{i} \\ {C}_{B} = \frac{1}{η} {\sum }_{i} {B}_{i}\end{matrix}$$

Now a matrix H is defined using the centroid and both matrices A and B as

$$H = ( A - {C}_{A} ) ( B - {C}_{B} )$$

Now single value decomposition of matrix H will be:

$$[ U , S , V ] = S V D ( H )$$

Now rotation matrix for points et A and B is defined as:

$$R = V {U}^{T}$$

These formulas are used to calculate rotation matrix which can be used for the alignment of points while verifying whether yaw is neutral or not. This is also done while performing exercises for extension and flexion.

Full Envelope of Movements can be traced with the system 100 to evaluate mandibular motion and all other physiological movements can be traced with precision.

When Flexion or Extension, or any other positioning, is taken with the use of an X-ray, it is important to record the positioning angle at which positioning the X-ray was taken for future re-positioning, if necessary. For this, a capture device 162 (e.g., connected to the processing device 106) can contain an X-ray sensor diode connected to the system's computer AI software so that when the X-ray energy is landing at the diode of the capture device 162, the capture device 162 transmits electrical signals from the capture device 162 to the AI software 106 while recording the patient's positioning degrees during X-rays taken at a particular patient's spatial positioning in space.

The system 100 has the two cameras positioned orthogonally and is able to position cervical spine in Neutral, Flexion, and Extension for Alteration of Motion Segment Integrity of Spine (AOMSI) studies. Positioning in orthogonal view for the purpose of minimizing optical distortion for precise AOMSI studies of X-ray images is a problem area and this is the problem solving technology. AOMSI calculation is a requirement under Guides to the Evaluation of Permanent Impairment, 6^{th} Edition that most of the States in USA, Canada, Australia, and New Zealand adopted. Based on the requirement, the quantification AOMSI report is used for disability ratings at the moment when maximum medical improvement is reached. AOMSI is a term used for findings in the spine when abnormal motion is diagnosed, and the findings are used for qualifications for permanency rating. Protocols describing various standards are published by the AMA. Only Spinal X-ray studies with pure orthogonal views are qualified for these types of biomechanical evaluation.

In one embodiment, the system 100 further includes vibration sensors 150. While jaw movements are performed, the vibration sensors 150, e.g., accelerometers, are positioned at as closest location to the TMJ disc projections as possible. The TMJ disc consists of a dense connective tissue and the tissue around it is highly vascularized. The synovial membrane provides nutrition and lubrication to the disc through synovial fluid. In a normal joint, there is little to no vibration that can be recorded when the joint is healthy. But, if and when the joint is diseased, or something happened to its supporting ligaments and the disc is in malposition, or desiccated, or inflamed, etc., then all kinds of vibrations occur inside of the joint while in motion. Depending on the nature of the vibrations, a diagnosis can be made.

In one embodiment, data from the vibration sensors 150 is captured simultaneously with images captured by the first and/or second cameras 102, 104. In this manner, the system and method of the present disclosure may identify with precision, and reliability at what point of opening and closing there is a vibration. Vibration is the point of a disc movement. When there are stretched ligaments inside the disk, the movements are increased in relation to a normal joint with normal ligaments. When there is a disc disease (15% of all arthritic patients report TMJ involvement), the disc creates a certain vibration. So, depending on the state of the supporting tissues and status of the disc the vibrations are different. Since TMJs are a paired joint, we are able to see vibrations on both sides and we can see which joint initiated the vibration and which one produced an “echo” as a mirror image of that initial vibration and at what height of opening and closing.

If there is a need to increase space between the upper and lower teeth, the change in temporomandibulomaxillary relationship ensues, and constructing permanent prosthesis with reproducible and reliable data records is extremely desirable. As for example, after TMD Phase 1 reversible medical procedures to control pain fails then TMD Phase 2, is usually initiated and includes repositioning of the temporomandibular condyles permanently via full prosthetic mouth reconstruction. Here, the system of the present disclosure is invaluable providing information from vibrations from the discs and temporomandibulomaxillary relationship for prosthetic reconstruction. The system allows to monitor changes in temporomandibulomaxillary relationship while monitoring changes in vibrations output. In certain TMD cases with, e.g. anterior disc displacement with reduction, Temporomandibular disc perforations, removal of vibrations indicates the condyle not to exerting pressure on retrodiscal tissues any longer, therefore, mitigating pain.

When people lose their teeth, they lose their Vertical Dimension of Occlusion because teeth maintain that vertical dimension. Maximum Intercaspal (MI) Position corresponds to the Lower Third of the Facial Height while teeth are at maximum intercaspation and is referred to Vertical Dimension of Occlusion (VDO). It changes over time in most people due to loss of tooth structure. Once the original VDO is lost, proprioceptive memory on how upper and lower jaws relate to each other is lost very shortly afterwards. The process of finding an acceptable VDO and Rest Space (RS) are paramount for proper functional maxillary/mandibular prosthetic fabrication. Improper VDO and mandibular-maxillary relationship can also cause problems for airway patency during sleep, which may be one of the causes of secondary Obstructive Sleep Apnea (OSA), which can cause by itself multitude chronic medical issues. Improper VDO causes increases in swallowing time, increases in muscle activity, etc., causing chronic pain and headaches. Both inadequate and enlarged VDO causes issues during speech distorting original sound phonetics. During prosthetic fabrication, dentists, subjectively, check on how the position of the artificial teeth in the upper or lower jaw, or both, allow the patient to produce different sounds. By modifying teeth's location and/or VDO during intermediate steps of prosthetic fabrication, doctors empirically via trial make sure their patients can also have ability to sound properly during speech. Unfortunately, mistakes can happen during fabrication process because this method is purely subjective and imprecise. It is especially true when plastic teeth are set on wax rims and the rims turned to plastic material which after processing undergoes dimensional changes. Or, with time, plastic teeth wear off against the opposite side. Moreover, with time, mandible and maxilla shrink and change creating further changes in VDO, therefore the changes are constant and dynamic. Therefore, phonetics of certain sounds are dynamic and interdependent on changes in dynamics of VDO.

One solution, for example, is to incorporate an audible capture device 152 in the system 100. During recording of the base data of the patient's temporomandibulomaxillary functional exercises, the system 100 also records initial patient's ability to pronounce certain sounds, with its sound quality recorded at a baseline VDO when normal teeth are still present. These records are available for retrieval by any dentist with patient' prior approval. Initial baseline data (e.g., from ROM, VDO, Lateral Movements, Lateral Canine Guidance, Anterior Incisal Guidance, Swallow Data, Rest Position, data from Electromyography, data from Vibration Sensors together with Patient Phonetics Record (PPR)) may be stored and can be successfully used for purposes to reconstruct most appropriate temporomandibulomaxillary relationship for most physiological dental prosthetic fabrications. Dentists would be retrieving initial records of different sounds pronounced by patients when the original VDO was recorded as a baseline. If a dentist needs to build a prosthesis for that patient, he will need to use the data from the system to re-establish the original positioning relationship of temporomandibulomaxillary relationship according to the relationship recorded in the past using the quality data of the sounds recorded at the baseline VDO. The sound quality record obtained by the audible capture device 152 would help dentists to find the most appropriate relationship of the jaws with respect to each other, with help of the TMJ system of the present disclosure, which can bring the same, or closest to the same VDO with respect to quality of sound recordings.

In use, baseline sound quality corresponds to the baseline intraoral volume of space. The space is filled with intraoral soft tissues and maintained with the bony structures. When the intraoral structures change over time or lost, the intraoral volume is changed and the relationship between them is also changed affecting speech. By evaluating the speech quality in relation to the baseline recorded earlier, the system is helping the doctor to be able to modify the speech quality to bring it as closest to the baseline as possible while modifying jaw relationship and intraoral volume. A dentist would modify the intraoral volume, positioning of artificial teeth in wax stage, changeable later to a permanent material in a dental lab, to relate the intraoral structures closest to that level as they were at the baseline for achieving that patient's baseline phonetics not subjectively, but with use of precise and reliable data from the system.

This relationship could be the most precise and closest to the original temporomandibulomaxillary relationship ever recorded with the original VDO when patients had their original teeth and jaw relationships. The clamp 108 allows the patient to position the mandible while teeth are not subject to any hindrance.

Once reversible treatment (Phase 1 TMD) has been performed and care necessitates to permanently establish lost, or new temporomandibulomaxillary relationship, a doctor can offer the Phase 2, irreversible reposition Temporomandibular Joints towards Skull with fixed dental prosthetics, and/or orthodontics. This technology can help to perform Phase 2 TMD treatment with precise control of temporomandibulomaxillary relationship and great results under strict controls of the records gathered from the past, during, and post-treatment and during each follow up process.

FIG. 20 is a block diagram illustrating physical components of a processing device 106, for example, a client computing device, a server, or any other computing device, with which examples of the present disclosure may be practiced. In a basic configuration, the processing device 106 may include at least one processing unit 1204 and a system memory 1206. Depending on the configuration and type of computing device, the system memory 1206 may comprise, but is not limited to, volatile storage (e.g., random access memory), non-volatile storage (e.g., read-only memory), flash memory, or any combination of such memories. The system memory 1206 may include an operating system 1207 and one or more program modules 1208 suitable for running software programs/modules 1220 such as 10 manager 1224, other utility 1226 and application 1228. As examples, system memory 1206 may store instructions for execution. Other examples of system memory 1206 may store data associated with applications. The operating system 1207, for example, may be suitable for controlling the operation of the processing device 106. Furthermore, examples of the present disclosure may be practiced in conjunction with a graphics library, other operating systems, or any other application program and is not limited to any particular application or system. This basic configuration is illustrated in FIG. 20 by those components within a dashed line 1222. The computing device 1106 may have additional features or functionality. For example, the processing device 106 may also include additional data storage devices (removable and/or non-removable) such as, for example, magnetic disks, optical disks, or tape. Such additional storage is illustrated in FIG. 20 by a removable storage device 1209 and a non-removable storage device 1210.

As stated above, a number of program modules and data files may be stored in the system memory 1206. While executing on the processing unit 1204, program modules 1208 (e.g., Input/Output (I/O) manager 1224, other utility 1226 and application 1228) may perform processes including, but not limited to, one or more of the stages of the operations described throughout this disclosure. For example, one such application 1228 may implement the facial recognition software and the facial points mapping software described in relation to FIGS. 5 and 7-19. Other program modules that may be used in accordance with examples of the present disclosure may include electronic mail and contacts applications, word processing applications, spreadsheet applications, database applications, slide presentation applications, drawing or computer-aided application programs, photo editing applications, authoring applications, etc. It is to be appreciated that several modules or applications 1228 may be execute simultaneously or near simultaneously and may share data.

Furthermore, examples of the present disclosure may be practiced in an electrical circuit comprising discrete electronic elements, packaged or integrated electronic chips containing logic gates, a circuit utilizing a microprocessor, or on a single chip containing electronic elements or microprocessors. For example, examples of the present disclosure may be practiced via a system-on-a-chip (SOC) where each or many of the components illustrated in FIG. 20 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality described herein may be operated via application-specific logic integrated with other components of the processing device 106 on the single integrated circuit (chip). Examples of the present disclosure may also be practiced using other technologies capable of performing logical operations such as, for example, AND, OR, and NOT, including but not limited to mechanical, optical, fluidic, and quantum technologies. In addition, examples of the present disclosure may be practiced within a general purpose computer or in any other circuits or systems.

The processing device 106 may also have one or more input device(s) 1212 such as a keyboard, a mouse, a pen, a sound input device, a device for voice input/recognition, a touch input device, etc. The output device(s) 1214 such as a display, speakers, a printer, etc. may also be included. The aforementioned devices are examples and others may be used. The processing device 106 may include one or more communication connections 1216 allowing communications with other computing devices 918 (e.g., external servers) and/or other devices of the positioning system such as energy emission device 164. Examples of suitable communication connections 1216 include, but are not limited to, a network interface card; RF transmitter, receiver, and/or transceiver circuitry; universal serial bus (USB), parallel, and/or serial ports; and/or wireless transceiver operating in accordance with, but not limited to, WIFI protocol, Bluetooth protocol, mesh-enabled protocol, etc.

The term computer readable media as used herein may include computer storage media. Computer storage media may include volatile and nonvolatile, removable and non-removable media implemented in any method or technology for storage of information, such as computer readable instructions, data structures, or program modules. The system memory 1206, the removable storage device 1209, and the non-removable storage device 1210 are all computer storage media examples (i.e., memory storage.) Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 1202. Any such computer storage media may be part of the computing device 1202. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

It is to be appreciated that the various features shown and described are interchangeable, that is a feature shown in one embodiment may be incorporated into another embodiment.

While the disclosure has been shown and described with reference to certain preferred embodiments thereof, it will be understood by those skilled in the art that various changes in form and detail may be made therein without departing from the spirit and scope of the disclosure.

Furthermore, although the foregoing text sets forth a detailed description of numerous embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

What is claimed is:

1. An apparatus comprising:
a clamp arranged in a fixed position with respect to a first bone of a patient's joint, the clamp having a reference object attached thereto;
one or more cameras arranged to capture images of facial landmark points and the reference object; and
a processing device configured to receive the captured images from the one or more cameras and monitor characteristics of movements and positioning of the first bone of the patient's joint with respect to a second bone of the patient's joint.

2. The apparatus of claim 1, wherein the patient's joint includes one or more Temporomandibular Joints (TMJs), the first bone of the patient's joint is a Mandible, the second bone affecting the patient's joint is a Maxilla, and the characteristics of movements and positioning of the first bone with respect to the second bone and one or more TMJ discs is a Temporomandibulomaxillary relationship.

3. The apparatus of claim 2, wherein the processing device is configured to create a baseline image including the facial landmark points.

4. The apparatus of claim 2, wherein the processing device is further configured to use characteristics of the Temporomandibulomaxillary relationship in the design of dental prosthetics for the patient and in diagnosis and treatment of Temporomandibular Disorders (TMD).

5. The apparatus of claim 1, wherein the reference object is a color-coded spherical object.

6. The apparatus of claim 1, wherein the clamp includes an upper prong, a shaft, and an adjustable lower prong, and wherein the clamp is configured to compress soft tissue surrounding the first bone to hold the clamp in a fixed relationship with the first bone.

7. The apparatus of claim 1, further comprising a display device connected to the processing device, the display device configured to present a User Interface (UI) that shows images captured by the one or more cameras.

8. The apparatus of claim 7, wherein the UI is further configured to provide one or more selectable buttons allowing a user to select options for operating the apparatus.

9. The apparatus of claim 1, wherein the processing device is configured to utilize Artificial Intelligence (AI) to monitor the characteristics of the movements and positioning of the first bone with respect to the second bone.

10. The apparatus of claim 1, further comprising one or more of a vibration sensor and an audible capture device for detecting one or more of vibration, sound, and voice at the patient's joint related to movement of the first bone with respect to the second bone to further characterize the patient's joint.

11. The apparatus of claim 1, wherein the processing device is further configured to create a homography matrix based on a current image plane with respect to a reference image plane.

12. The apparatus of claim 1, wherein the processing device is further configured to utilize the characteristics of the movements and positioning of the first bone with respect to the second bone to create one or more graphs and contours.

13. A method comprising the steps of:
arranging a clamp in a fixed position with respect to a first bone of a patient's joint, the clamp having a reference object attached thereto;
capturing images of facial landmark points and the reference object; and
receiving the captured images and monitoring characteristics of movements and positioning of the patient's TMJ joints.

14. The method of claim 13, wherein the patient's joint includes one or more Temporomandibular Joints (TMJs), the first bone of the patient's joint is a mandible, the second bone of the patient's joint is a maxilla, and the characteristics of movements and positioning of the first bone with respect to the second bone is a temporomandibulomaxillary relationship.

15. The method of claim 14, further comprising one or more of the steps of:
creating a baseline image including the facial landmark points; and
using characteristics of the temporomandibulomaxillary relationship in the design of dental prosthetics for the patient and in diagnosis and treatment of Temporomandibular Disorders (TMD).

16. The method of claim 13, further comprising the step of presenting a User Interface (UI) that shows the captured images and one or more selectable buttons.

17. The method of claim 13, further comprising the step of utilizing Artificial Intelligence (AI) to monitor the characteristics of the movements and positioning of the first bone with respect to the second bone.

18. The method of claim 13, further comprising the step of detecting audible signals at the patient's joint related to movement of the first bone with respect to the second bone to further characterize the patient's joint.

19. The method of claim 13, further comprising the step of creating a homography matrix based on a current image plane with respect to a reference image plane.

20. The method of claim 13, further comprising the step of utilizing the characteristics of the movements and positioning of the first bone with respect to the second bone to create one or more graphs and contours.

21. The apparatus of claim 10, wherein the audible capture device captures audio recordings of the patient in relation to the temporomandibulomaxillary relationship for baseline reference.

22. An apparatus comprising:
at least two cameras arranged to capture images of a head of a patient, a first camera of the at least two cameras arranged toward a front of the head of the patient and a second camera of the at least two cameras toward a side of the head of the patient; and
a processing device configured to:
receive the captured images from at least two cameras,
detect facial landmarks from the captured images, and
determine movements and positioning of at least one point of the head of the patient based on the detected facial landmarks.

23. The apparatus of claim 22, wherein the processing device is further configured to create a homography matrix based on a current image plane with respect to a reference image plane.

24. The apparatus of claim 23, wherein the movements and positioning of a first portion of the patient relative to a second portion of the patient include flexion and extension.

25. The apparatus of claim 22, further comprising an X-ray sensor diode coupled to the processing device so that when X-ray energy is landing at the X-ray sensor diode an electrical signal is transmitted to the processing device to record the patient's positioning degrees during X-rays taken at a particular patient's spatial positioning in space.

26. The apparatus of claim 22, wherein the first and second cameras are arranged orthogonally to each other.

27. The apparatus of claim 22, wherein the processing device determines the movements and positioning of the patient using an artificial intelligence (AI) function based on the detected landmarks.

28. The apparatus of claim 27, wherein the processing device determines the movements and positioning of at least a cervicocranium and/or neck of the patient.

29. The apparatus of claim 22, wherein the detected facial landmarks include at least a tip of the patient's nose and the processing device generates a horizontal line to an ear of the patient, where the horizontal line is used to determine a zero pitch of the head of the patient.

30. The apparatus of claim 22, further comprising an energy emission device, wherein the processing device is further configured to selectively activate the energy emission device when a position of the head of the patient matches a predetermined position.

31. The apparatus of claim 30, wherein the energy emission device is an ionizing high energy generating device.

32. An apparatus comprising:
at least one camera arranged to capture images of a head of a patient; and
a processing device configured to:
receive the captured images from the at least one camera,
detect facial landmarks from the captured images, and
determine movements and positioning of at least one point of the head of the patient based on the detected facial landmarks; and
an X-ray sensor diode coupled to the processing device so that when X-ray energy is landing at the X-ray sensor diode an electrical signal is transmitted to the processing device to record the patient's positioning degrees during X-rays taken at a particular patient's spatial positioning in space.
